(19) United States
(12) Patent Application Publication
(10) Pub. No.: US 2018/0125377 A1
(43) Pub. Date: May 10, 2018

(54) BLOOD PRESSURE MEASUREMENT DEVICE, BLOOD PRESSURE MEASUREMENT METHOD, AND RECORDING MEDIUM

(71) Applicant: NEC Corporation, Minato-ku, Tokyo (JP)

(72) Inventors: Kimiyasu TAKOH, Tokyo (JP); Katsumi ABE, Tokyo (JP); Masahiro KUBO, Tokyo (JP); Yuji OHNO, Tokyo (JP); Ersin ALTINTAS, Tokyo (JP); Takeshi AKAGAWA, Tokyo (JP); Tetsuri ARIYAMA, Tokyo (JP)

(73) Assignee: NEC Corporation, Minato-ku, Tokyo (JP)

(21) Appl. No.: 15/573,532

(22) PCT Filed: May 20, 2016
(86) PCT No.: PCT/JP2016/002460

§ 371 Date: Nov. 13, 2017

(30) Foreign Application Priority Data
May 28, 2015 (JP) 2015-108033

(51) Int. Cl. A61B 5/0225; A61B 5/022

(52) U.S. Cl. CPC A61B 5/0225; A61B 5/02208

(57) ABSTRACT

A blood pressure measurement device estimates blood pressure based on first internal pressure of cuff during a first period and Korotkoff sound during the first period or based on the first internal pressure during the first period and first pulse wave during the first period; calculates timings when the first pulse wave satisfies a predetermined condition, a third period between timings, and pressure of first internal pressure during the third period and generates first information where the third period and the pressure are associated; generates blood pressure information where the calculated first information and the estimated blood pressure are associated; and specifies the first information matching or resembling second information generated based on second internal pressure of the cuff during second period and second pulse wave during the second period, and estimates the blood pressure associated with the specified first information as blood pressure during the second period.

### TECHNICAL FIELD

The present invention relates to a blood pressure measurement device and the like that estimate blood pressure.

### BACKGROUND ART

As a method for measuring blood pressure of a living body in a Non-Invasive manner, there is widely used a method in which a pressure unit such as a cuff or the like is set on a specific region of a living body, and an artery and a circumference of it are pressurized by the pressure unit to measure blood pressure. As blood pressure measurement devices that measure blood pressure in a Non-Invasive manner, there are devices such as a blood pressure measurement device based on a microphone method for detecting the Korotkoff sound using a microphone, and a blood pressure measurement device based on an oscillometric method.

These blood pressure measurement devices stop a blood flow in an artery in a specific region (measurement region) and thereby measure a systolic blood pressure that is blood pressure in a course of heart contraction. Therefore, it is necessary for the pressure unit to apply, to the artery, a pressure higher than systolic blood pressure (a systolic blood pressure value, maximum blood pressure, or Systolic blood pressure, hereinafter, described also as an “SBP”). However, pressure applied by the pressure unit is frequently a burden on a body during measurement.

To reduce the burden, PTL 1 or PTL 2, for example, discloses a blood pressure measurement device that reduces the pressure for measurement.

PTL 1 discloses a blood pressure measurement device capable of measuring blood pressure without using a pressure unit. The blood pressure measurement device calculates a characteristic value associated with blood pressure on the basis of a pulse wave measured under a non-pressure condition and estimates blood pressure on the basis of a correlation between the calculated characteristic value and a blood pressure value.

Further, PTL 2 discloses a blood pressure measurement device that measures systolic blood pressure on the basis of a wave height value of a pulse wave by using a cuff. The blood pressure measurement device estimates a systolic blood pressure via coefficient transformation of a wave height value of a pulse wave measured at internal pressure of the cuff lower than systolic blood pressure.

### CITATION LIST

### Patent Literature

PTL 1: Japanese Laid-open Patent Publication No. H10(1998)-295657

PTL 2: Japanese Laid-open Patent Publication No. 2003-111737

### SUMMARY OF INVENTION

### Technical Problem

A correlation between a characteristic value and blood pressure is affected by various factors such as elasticity of an artery and a diameter of the artery. In other words, even a correlation calculated for a certain condition is not always a correlation established for another condition. Since the blood pressure measurement device disclosed by PTL 1 estimates blood pressure on the basis of a particular correlation, the estimated blood pressure is not always accurate.

On the other hand, measuring a factor affecting accuracy for the correlation and maintaining the accuracy by correcting a correlation equation on the basis of the factor is known. However, for example, an ultrasound measurement device, a pulse wave propagation speed measurement device, or the like is required for measuring the factor. Therefore, a configuration of a device for estimating blood pressure on the basis of a correlation may be complicated or data processing may be cumbersome.

The blood pressure measurement device disclosed by the PTL 2 estimates blood pressure on the basis of an assumption in which an extent of a change in volume of an artery measured using a cuff is similar to an extent of a change in pressure in the artery. This assumption is established when extensibility of the artery is constant (or substantially constant) in the same manner as in a spring. However, with an increase of pressure, the extensibility of the artery decreases. Therefore, the above-described assumption does not become established as a pressure in the artery increases.

Further, a wave height value is fluctuated in accordance with a condition between a cuff and an artery, and is therefore markedly affected by body movements in a subject to be measured. Hence, it is difficult to measure the wave height value with high reproducibility. Thus, it is difficult to accurately estimate a systolic blood pressure on the basis of a wave height value.

Therefore, it is difficult for the blood pressure measurement devices disclosed by PTL 1 and PTL 2 to accurately estimate blood pressure.

Accordingly, a main object of the present invention is to provide a blood pressure measurement device and the like that estimate blood pressure with high accuracy.

### Solution to Problem

As an aspect of the present invention, a blood pressure measurement device including:

first blood pressure estimation means for estimating blood pressure on basis of first pressure signal indicating internal pressure of cuff during a first period and Korotkoff sound during the first period or on basis of the first pressure signal during the first period and first pulse wave signal indicating pulse wave during the first period;

pulse wave calculation means for calculating a plurality of timings when the first pulse wave signal satisfies a predetermined condition, a third period between the plurality of timings, and pressure of first pressure signal during the third period and generating first pulse wave information where the third period and the pressure are associated;

blood pressure information generation means for generating blood pressure information where the calculated first pulse wave information and the estimated blood pressure are associated with each other; and

second blood pressure estimation means for specifying the first pulse wave information matching or resembling second pulse wave information generated based on second pressure signal indicating internal pressure of the cuff during second period and second pulse wave signal indicating pulse wave during the second period, and estimating the blood pressure associated with the specified first pulse wave information as blood pressure during the second period.

In addition, as another aspect of the present invention, a blood pressure measurement method including:

estimating blood pressure on basis of first pressure signal indicating internal pressure of cuff during a first period and Korotkoff sound during the first period or on basis of the first pressure signal during the first period and first pulse wave signal indicating pulse wave during the first period;

calculating a plurality of timings when the first pulse wave signal satisfies a predetermined condition, a third period between the plurality of timings, and pressure of first pressure signal during the third period and generating first pulse wave information where the third period and the pressure are associated;

generating blood pressure information where the calculated first pulse wave information and the estimated blood pressure are associated with each other; and

specifying the first pulse wave information matching or resembling second pulse wave information generated based on second pressure signal indicating internal pressure of the cuff during second period and second pulse wave signal indicating pulse wave during the second period, and estimating the blood pressure associated with the specified first pulse wave information as blood pressure during the second period.

Furthermore, the object is also realized by a blood pressure estimation program, and a computer-readable recording medium that records the program.

### Advantageous Effects of Invention

According to the blood pressure measurement device and the like according to the present invention, blood pressure can be estimated with a high accuracy.

### BRIEF DESCRIPTION OF DRAWINGS

### Description of Embodiments

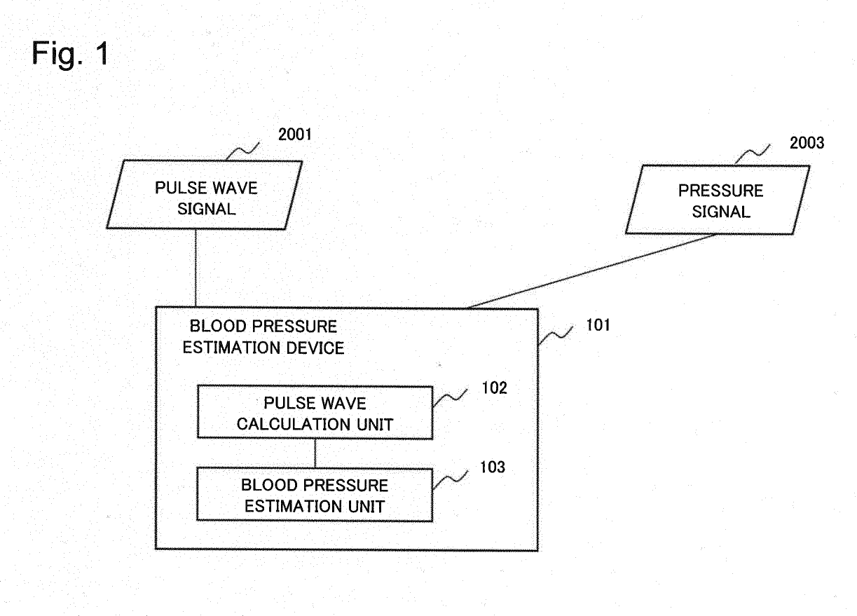

FIG. 1 is a block diagram illustrating a configuration of a blood pressure estimation device according to a first example embodiment of the present invention.

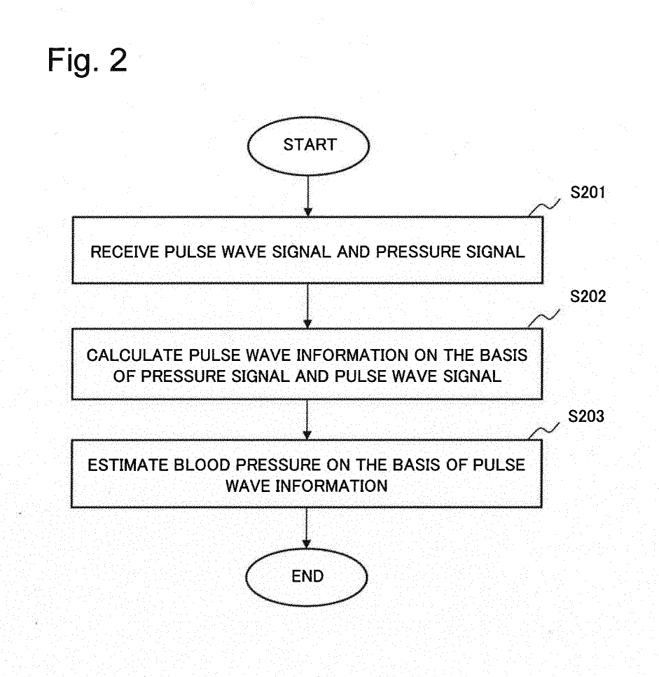

FIG. 2 is a flowchart illustrating a flow of processing in the blood pressure estimation device according to the first example embodiment.

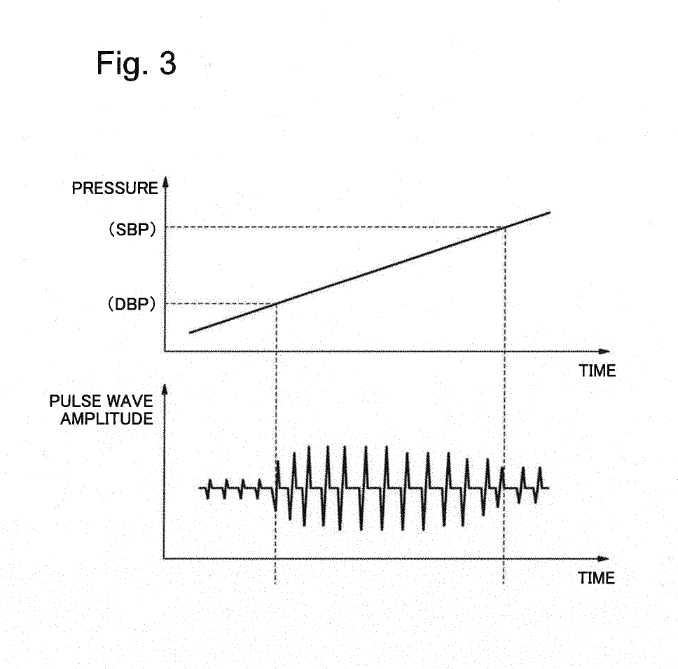

FIG. 3 is a diagram conceptually illustrating one example of a pressure signal received by the blood pressure estimation device.

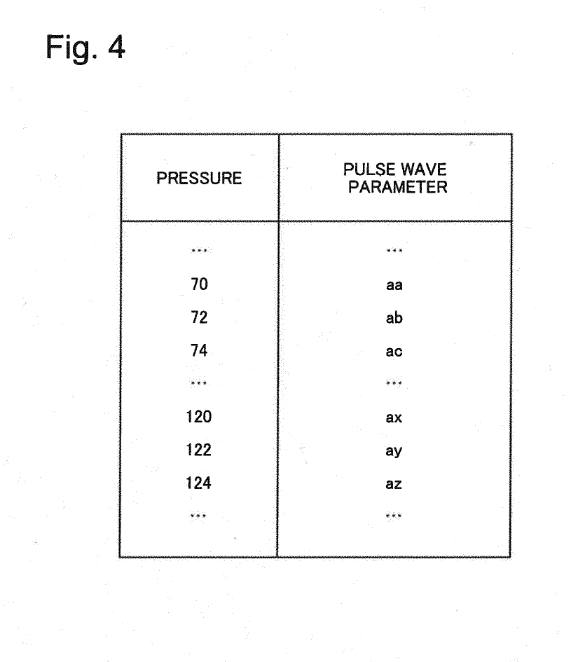

FIG. 4 is a diagram conceptually illustrating one example of a pulse wave information.

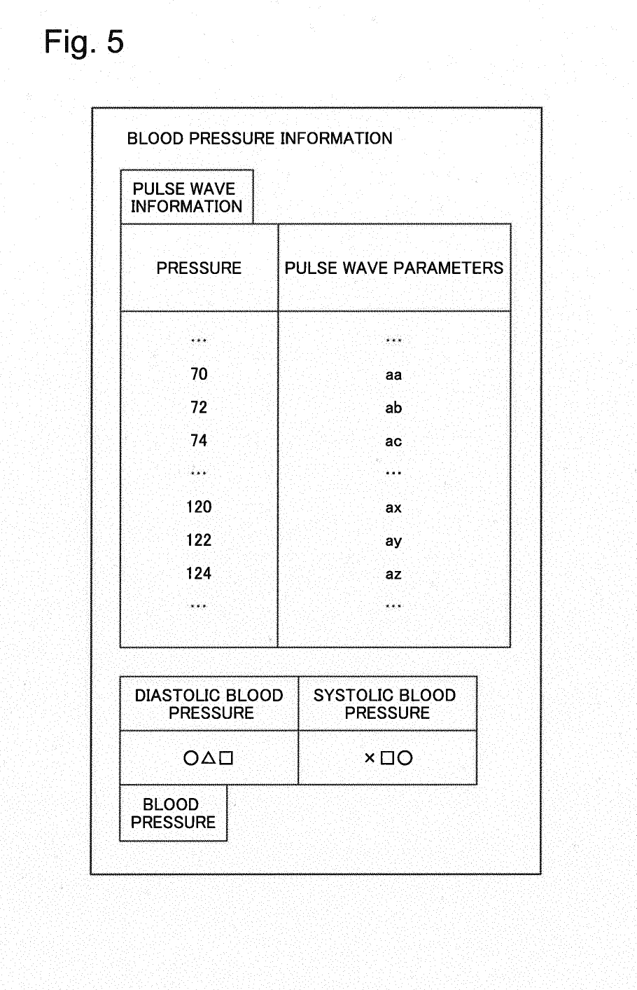

FIG. 5 is a diagram conceptually illustrating one example of blood pressure information.

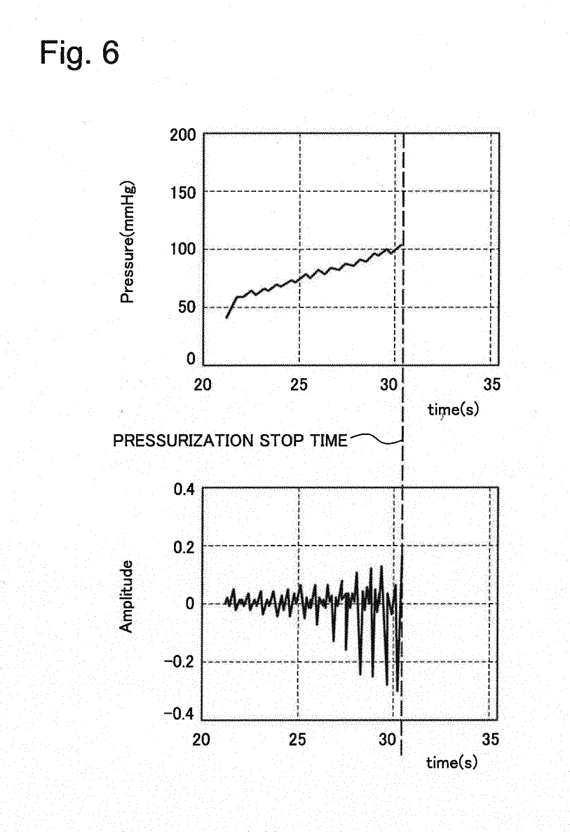

FIG. 6 is a diagram illustrating one example in which a range where a pressure signal fluctuates does not include systolic blood pressure.

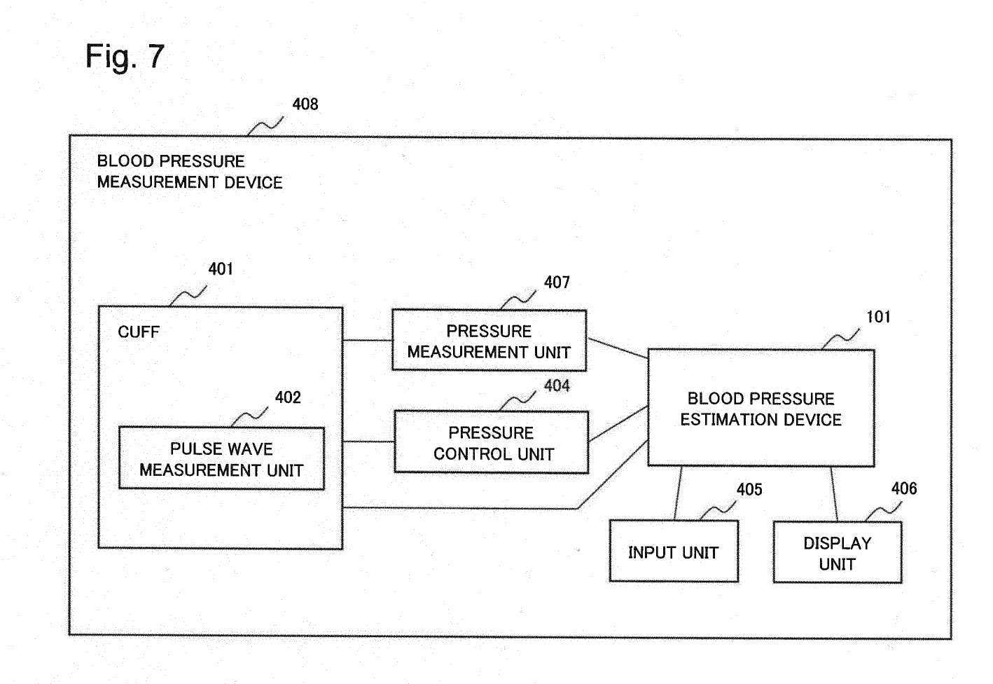

FIG. 7 is a block diagram illustrating a configuration of a blood pressure measurement device according to the first example embodiment.

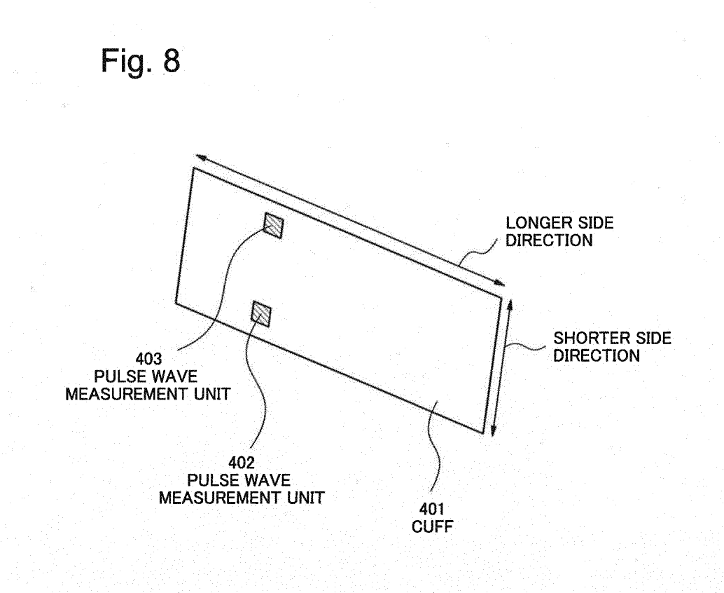

FIG. 8 is a perspective view of a cuff that is not placed.

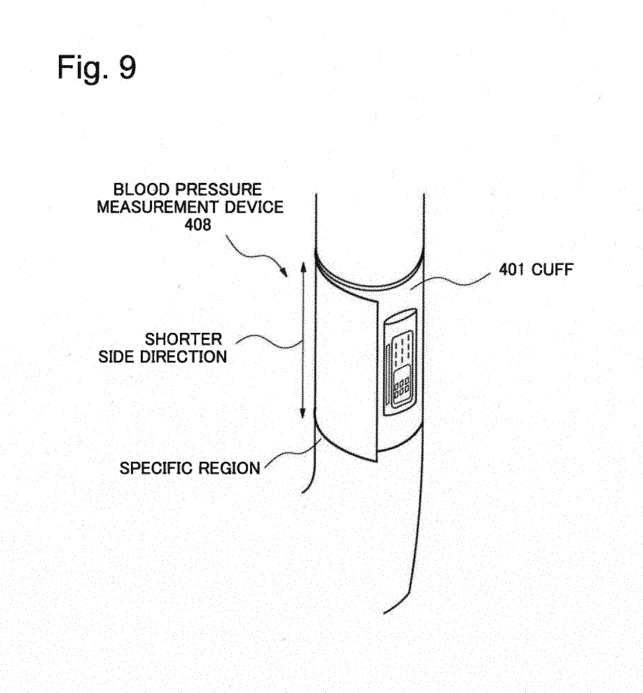

FIG. 9 is a diagram illustrating one example of a state where a cuff is placed on a specific region.

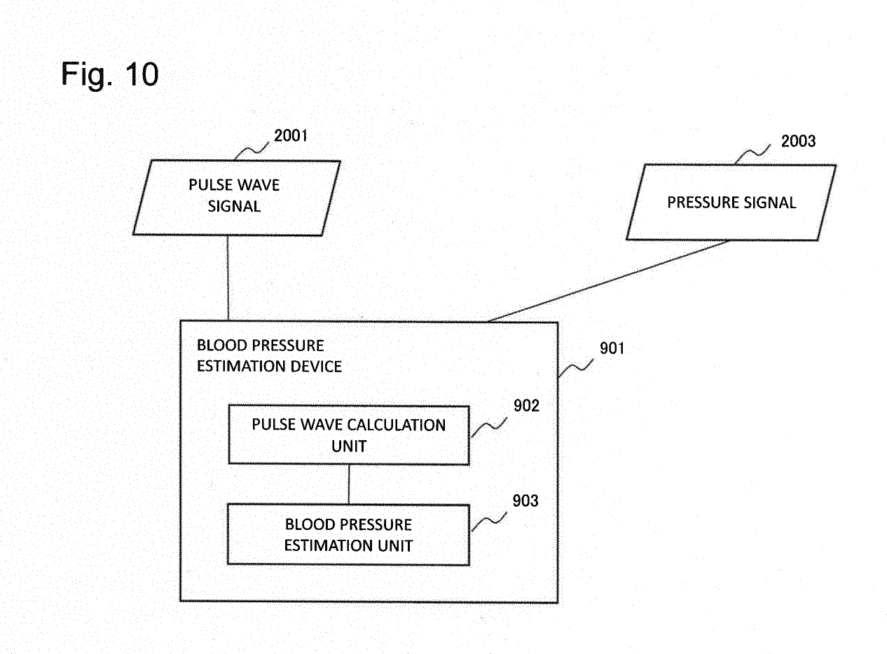

FIG. 10 is a block diagram illustrating a configuration of a blood pressure estimation device according to a second example embodiment of the present invention.

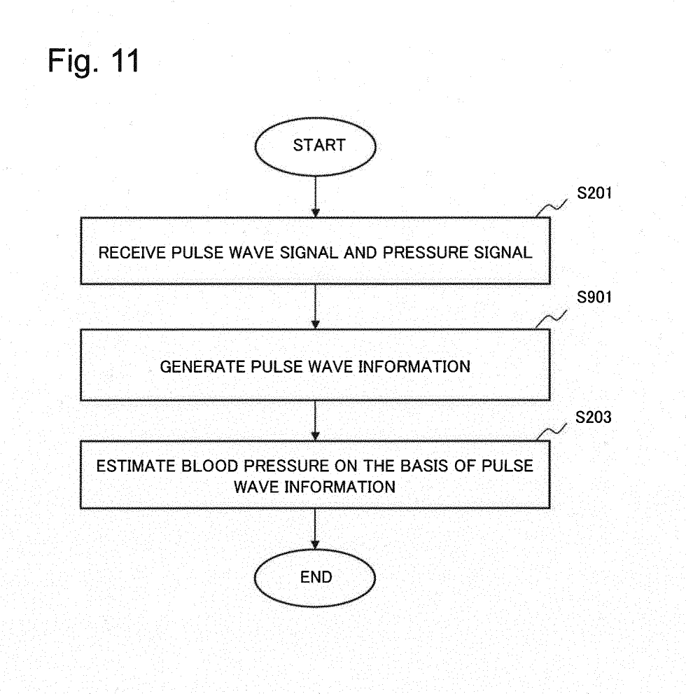

FIG. 11 is a flowchart illustrating a flow of processing in the blood pressure estimation device according to the second example embodiment.

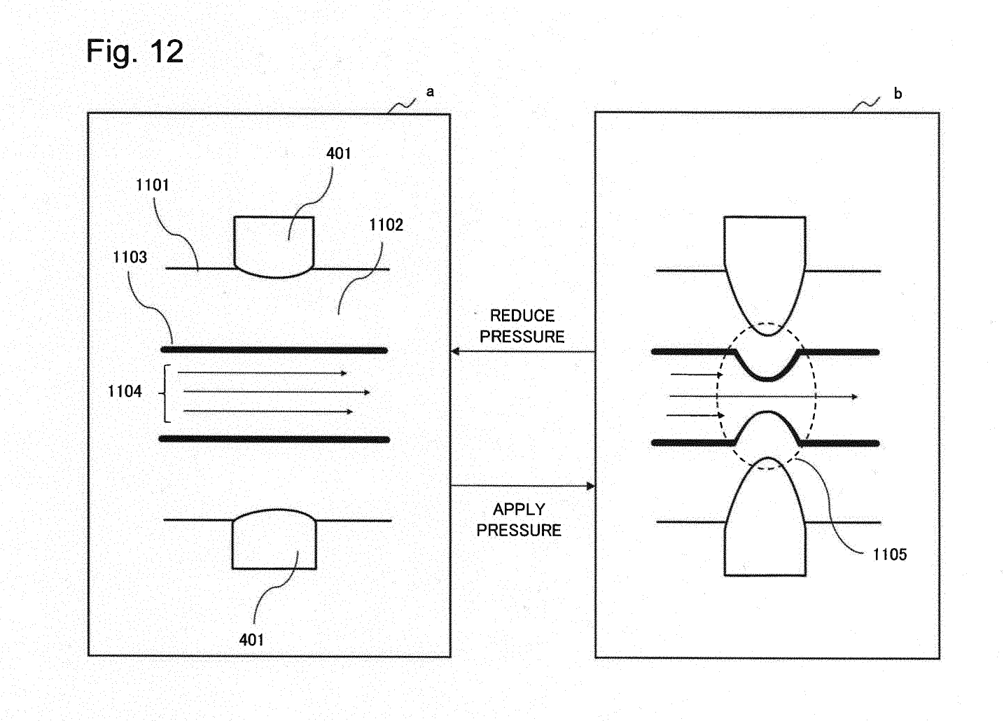

FIG. 12 is a cross-sectional view schematically illustrating a pressure signal and a specific region where a pulse wave signal is measured.

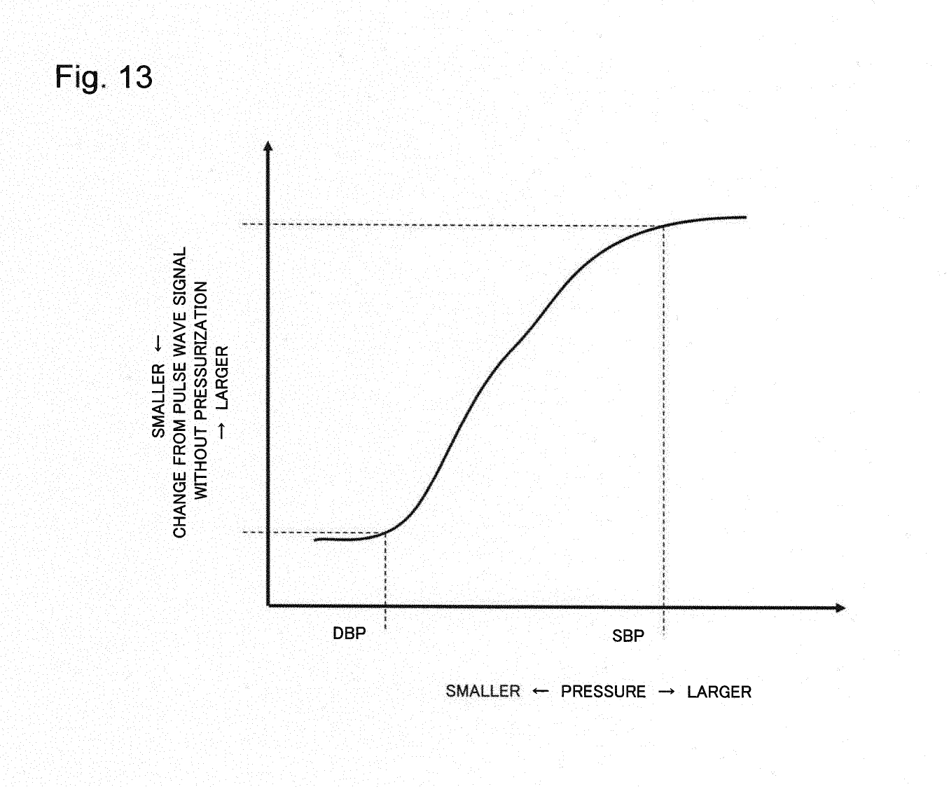

FIG. 13 is a diagram conceptually illustrating one example of a relation between a pressure signal and a plurality of pulse wave signals.

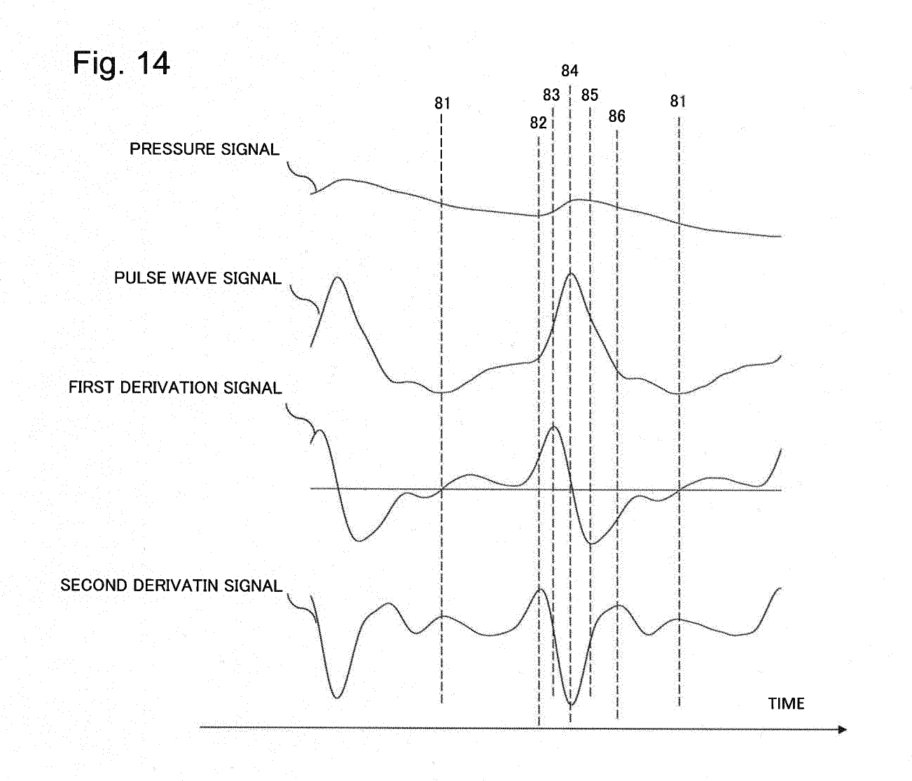

FIG. 14 is a diagram conceptually illustrating one example of processing for extracting a timing.

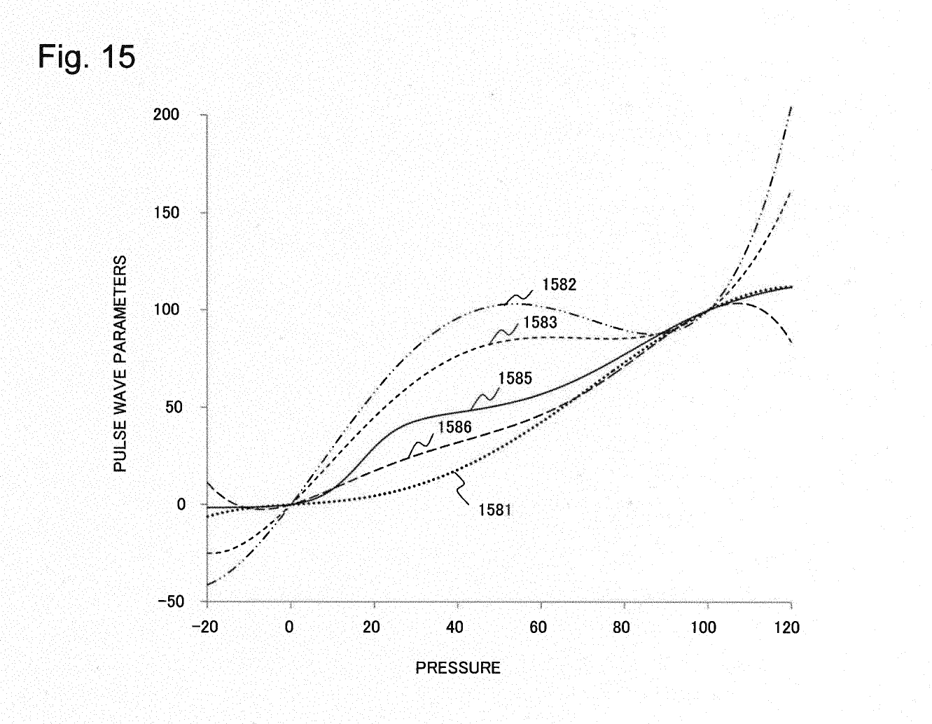

FIG. 15 is a diagram conceptually illustrating characteristics included in pulse wave information.

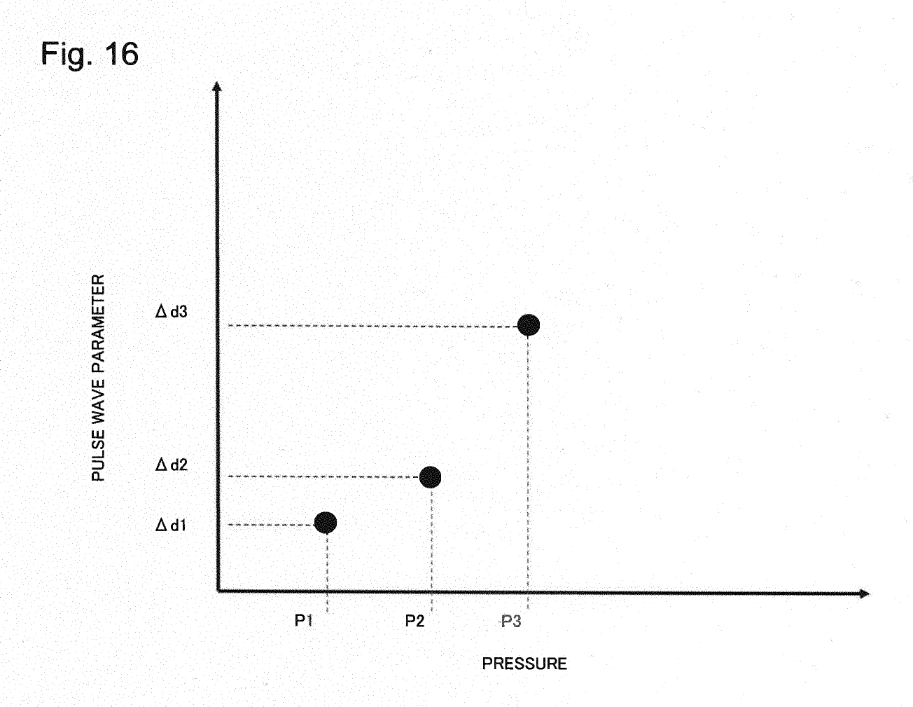

FIG. 16 is a diagram conceptually illustrating one example of a relation between a pressure signal and a difference signal in a case of an increase in pressure.

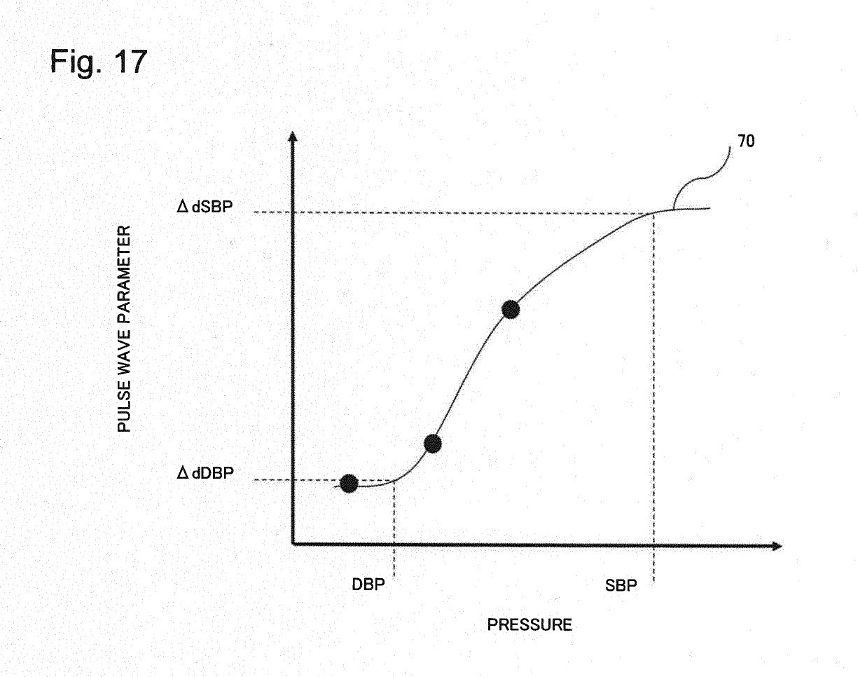

FIG. 17 is a diagram conceptually illustrating an example in which a curve representing a relation between a pressure signal and a difference signal is estimated.

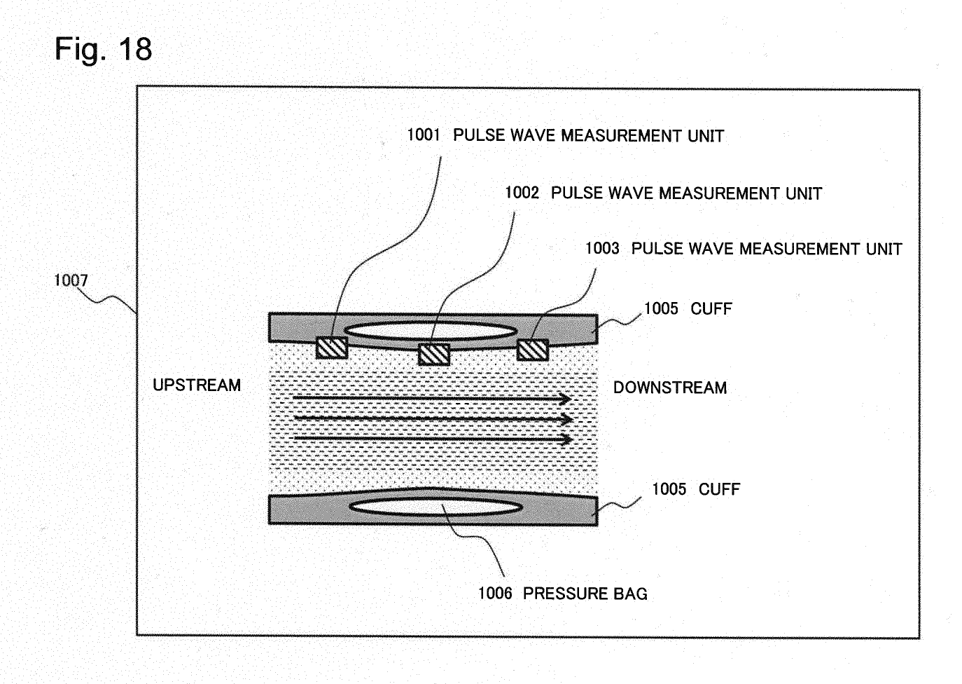

FIG. 18 is a diagram schematically illustrating a positional relationship between a cuff and three pulse wave measurement units.

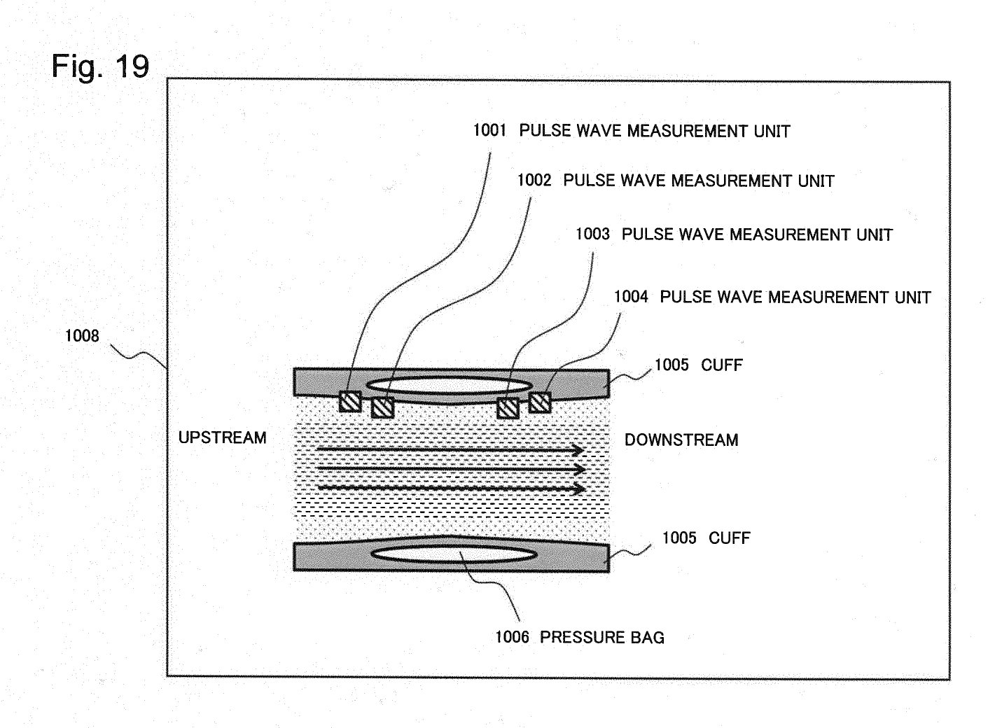

FIG. 19 is a diagram conceptually illustrating a position relation between a cuff and four pulse wave measurement units.

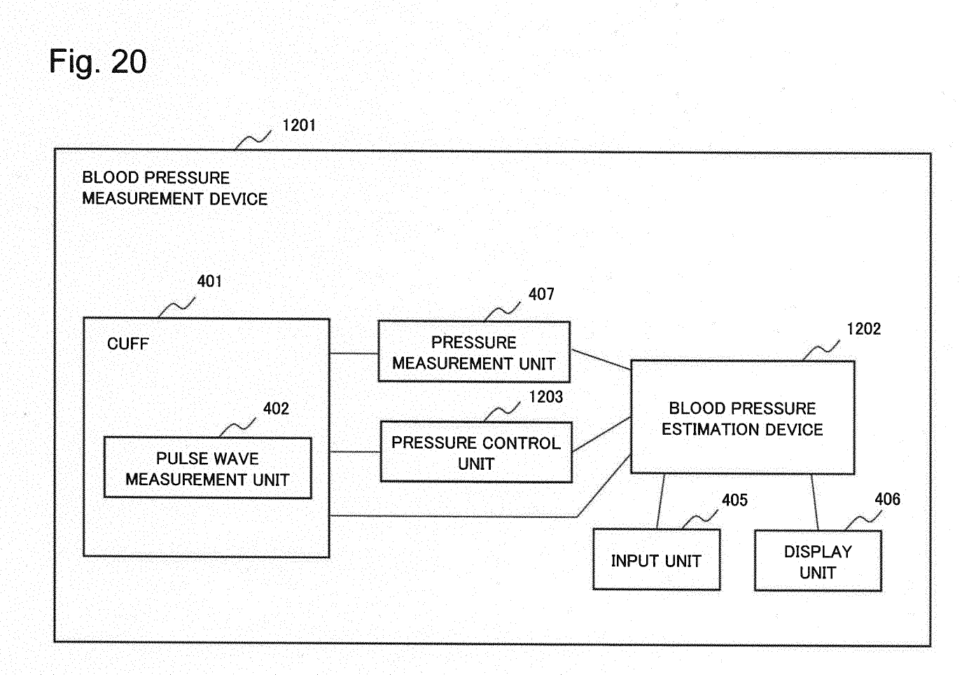

FIG. 20 is a block diagram illustrating a configuration of a blood pressure measurement device according to a third example embodiment of the present invention.

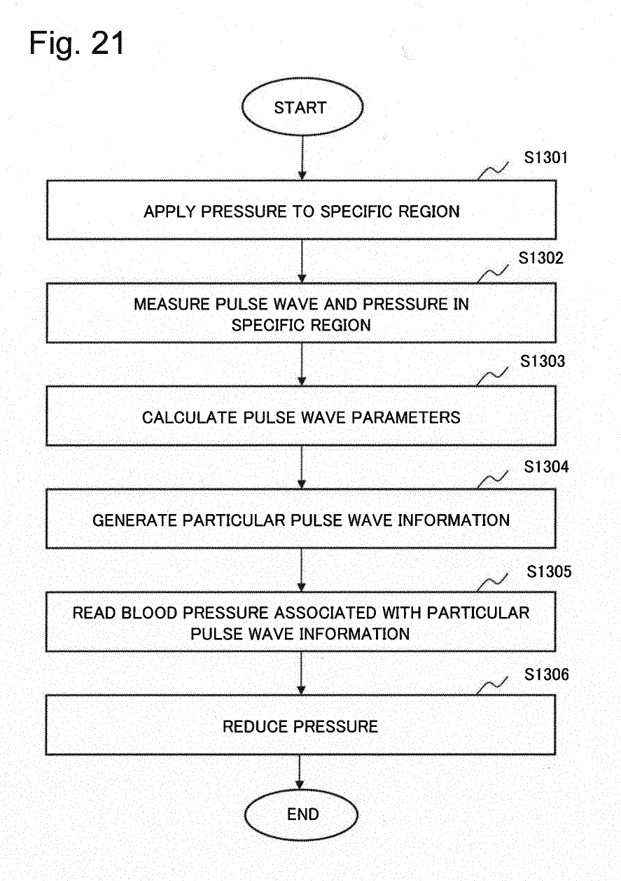

FIG. 21 is a flowchart illustrating a flow of processing in the blood pressure measurement device according to the third example embodiment.

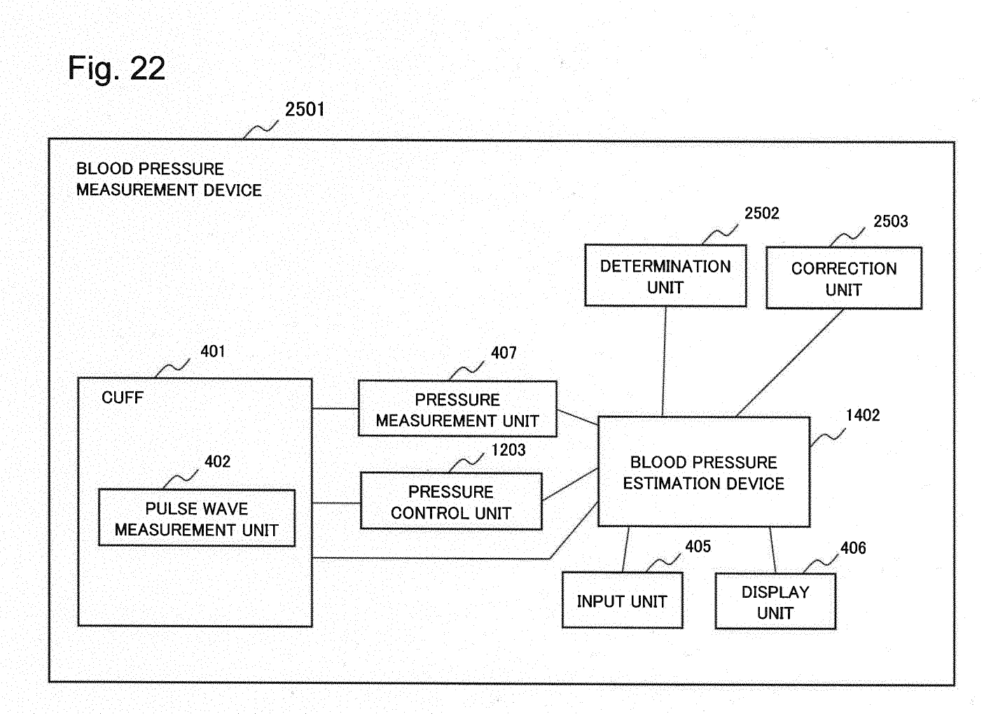

FIG. 22 is a block diagram illustrating a configuration of a blood pressure measurement device according to a fourth example embodiment of the present invention.

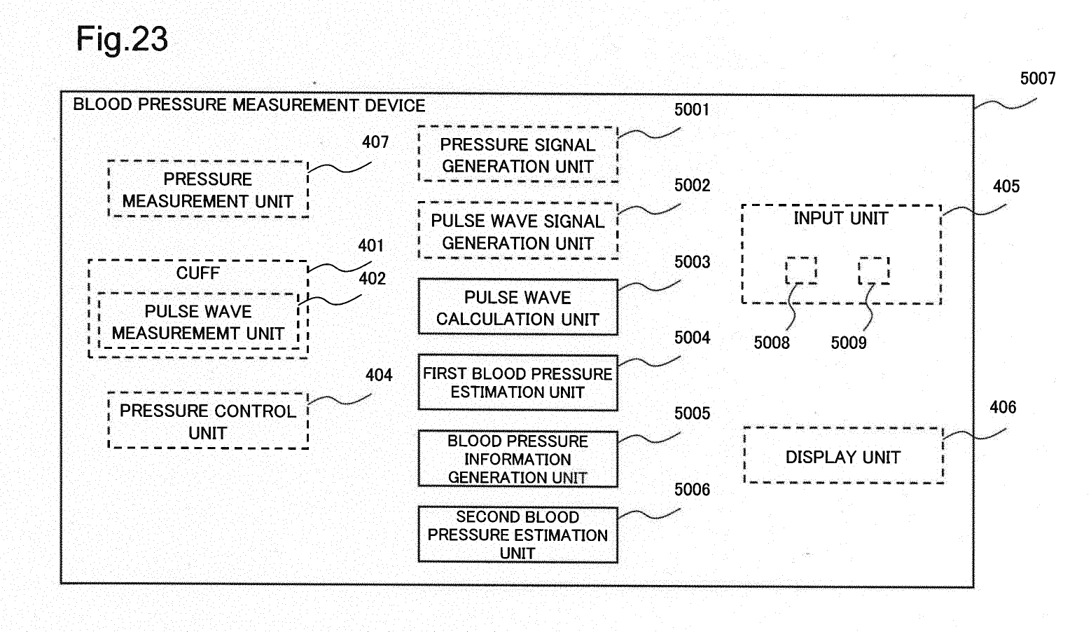

FIG. 23 is a block diagram illustrating a configuration of a blood pressure measurement device according to a fifth example embodiment of the present invention.

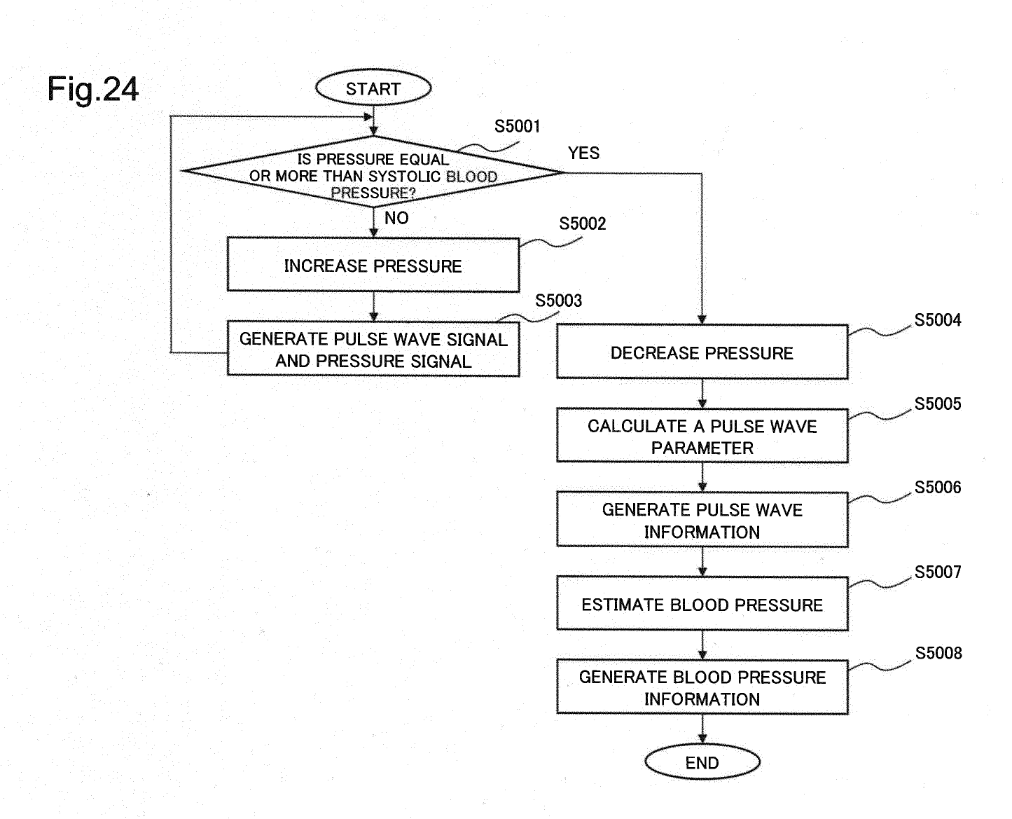

FIG. 24 is a flowchart illustrating a flow of processing in the blood pressure measurement device according to the fifth example embodiment.

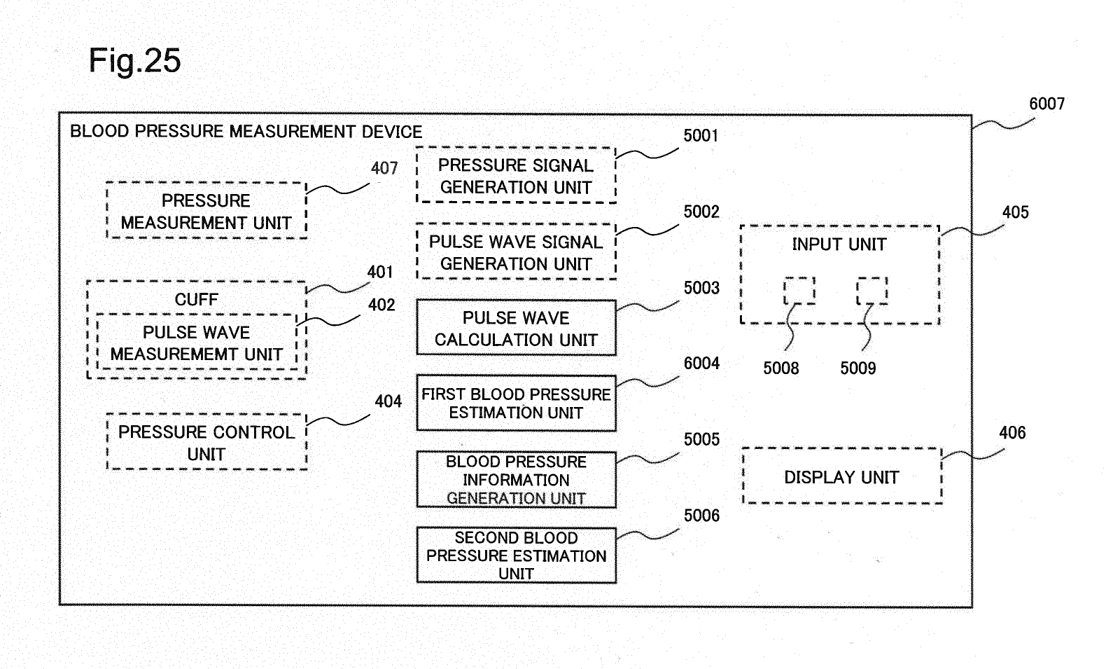

FIG. 25 is a block diagram illustrating a configuration of a blood pressure measurement device according to a sixth example embodiment of the present invention.

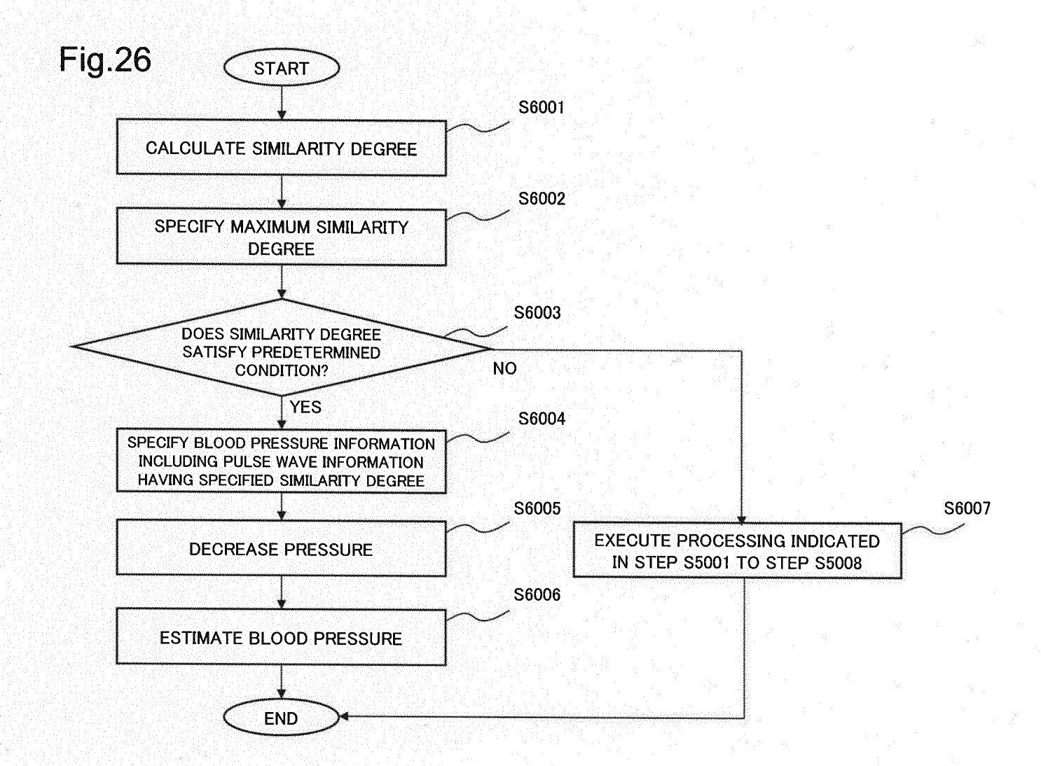

FIG. 26 is a flowchart illustrating a flow of processing in the blood pressure measurement device according to the sixth example embodiment.

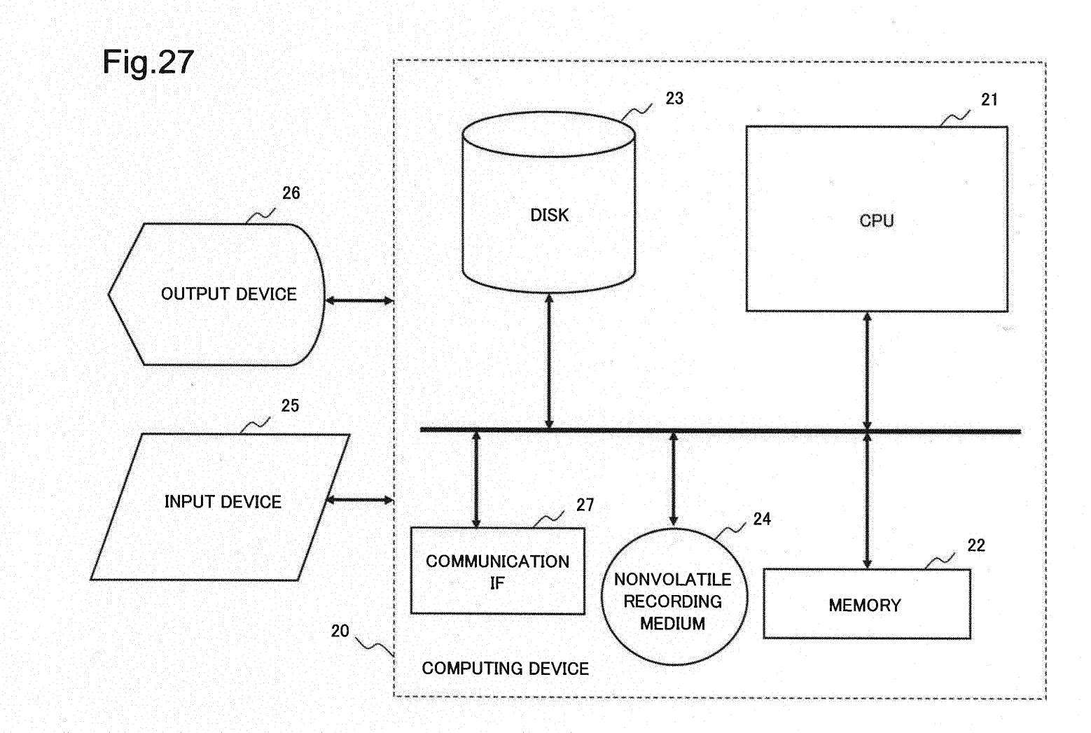

FIG. 27 is a block diagram schematically illustrating a hardware configuration of a calculation processing apparatus capable of realizing a blood pressure estimation device according to each example embodiment of the present invention.

Next, example embodiments of the present invention will be described in detail with reference to the drawings.

### FIRST EXAMPLE EMBODIMENT

A configuration of a blood pressure estimation device 101 according to a first example embodiment of the present invention and processing executed by the blood pressure estimation device 101 will be described in detail with reference to FIG. 1 and FIG. 2. FIG. 1 is a block diagram illustrating the configuration of the blood pressure estimation device 101 according to the first example embodiment of the present invention. FIG. 2 is a flowchart illustrating a flow of processing in the blood pressure estimation device 101 according to the first example embodiment.

The blood pressure estimation device 101 according to the first example embodiment includes a pulse wave calculation unit 102 and a blood pressure estimation unit 103.

The blood pressure estimation device 101 receives a pressure signal 2003 representing pressure in a certain time period and one or more pulse wave signals (e.g. pulse wave signals 2001) measured while the pressure is applied to a subject to be measured in the certain time period (step S201).

With reference to FIG. 3, one example of the pressure signal 2003 and a pulse wave signal 2001 received by the blood pressure estimation device 101 will be described. FIG. 3 is a diagram conceptually illustrating one example of the pressure signal 2003 and the pulse wave signal. The horizontal axis of FIG. 3 represents time and represents later time toward a rightward side. The vertical axis in the upper figure of FIG. 3 represents an amplitude of a pressure signal and represents that the amplitude of the pressure signal is stronger toward the upper side. The vertical axis in the lower figure of FIG. 3 represents an amplitude of a pulse wave signal and represents that the amplitude of the pulse wave signal increases closer to the upper end or the lower end, and the amplitude of the pulse wave signal decreases closer to a center of the upper end and the lower end. In the example illustrated in FIG. 3, the certain time period refers to a (heartbeat) period in which a heart beats at multiple times.

In the following description, for convenience of explanation, it is assumed that a cuff shape a is a rectangle (rectangular shape) while being developed as exemplified in FIG. 8 to be described later. It is assumed that a longer side direction is a direction where the cuff is wound around a specific region. Further, it is assumed that a shorter side direction is a direction orthogonal (or substantially orthogonal) to the longer side direction. Further, it is assumed that the entire cuff applies a pressure to the specific region in a state of pressurization. In this case, it is assumed that an “upstream” represents a portion between the nerve center or a heart and the center of the shorter side direction in an artery. It is assumed that a “downstream” represents a portion between the center of the shorter side direction and a peripheral side (e.g. a hand or foot) in the artery. However, an aspect of the cuff is not limited to the above-described manner.

The example illustrated in FIG. 3 represents a pulse wave signal 2001 measured while a pressure is applied at a constant (or substantially constant) rate in a certain time period. The pulse wave signal 2001 refers to, for example, a pulse wave signal measured in an upstream. The pulse wave signal 2001 may be a pulse wave signal measured in a downstream or a pulse wave signal measured in a center (or substantially in a center) of an area applied with a pressure.

Therefore, the pulse wave signal 2001 is a signal where an amplitude of the pulse wave and a timing of measuring the pulse wave are associated with each other. The pressure signal 2003 is a signal where an amplitude of a pressure and a timing of measuring the pressure are associated with each other.

Hereinafter, for convenience of explanation, it is assumed that one or more pulse wave signals are one pulse wave signal (i.e. a pulse wave signal 2001). A pulse wave signal received by the blood pressure estimation device 101 according to the present example embodiment may be two or more pulse wave signals.

Next, the pulse wave calculation unit 102 generates pulse wave information on the basis of the received pressure signal 2003 and the pulse wave signal 2001 (step S202). The pulse wave calculation unit 102 calculates, for example, a timing when the pulse wave signal 2001 satisfies a predetermined condition, also calculates a period representing a difference between a plurality of timings, and further calculates a value (i.e. pressure value) of the pressure signal 2003 in the period. The pulse wave calculation unit 102 calculates timings, periods, and pressure values in the periods for a plurality of predetermined conditions, respectively.

The pulse wave calculation unit 102 may calculate an average of a pressure signal 2003 in the period and thereby determine a pressure value in the period, or may determine a pressure value on the basis of a pressure based on a pressure signal 2003 at a certain timing in the period. A method of calculating a pressure value in the pulse wave calculation unit 102 is not limited to the above-described examples.

The predetermined condition is, for example, a condition that the pulse wave signal 2001 is the smallest (or around the smallest) in one heartbeat or is, for example, a condition that the pulse wave signal 2001 is the largest (or around the largest) in one heartbeat.

When there are multiple pulse wave signals 2001, a timing when a difference signal representing a difference among the pulse wave signals satisfies a predetermined condition may be calculated.

For example, “around the largest” can be defined as a value that is within a certain range from the largest. The certain range may be a predetermined value or a value calculated on the basis of a fact in which a magnitude of an inclination (determined by calculating a differential, a difference, or the like) of a target (e.g. the above-described pulse wave signal 2001) for which a largest value is calculated is less than a predetermined value. The certain range is not limited to the above-described examples.

Similarly, “around the smallest” can be defined as a value that is within a certain range from the smallest. The certain range may be a predetermined value or a value calculated on the basis of a fact in which a magnitude of an inclination (determined by calculating a differential, a difference, or the like) of a target (e.g. the above-described pulse wave signal 2001) for which a smallest value is calculated is less than a predetermined value. The certain range is not limited to the above-described examples.

For convenience of explanation, a timing of the smallest (or approximately smallest) pulse wave signal 2001 in one heartbeat is expressed as a “first timing.” Further, a timing of the largest (or approximately largest) pulse wave signal 2001 in one heartbeat is expressed as a “fourth timing.”

When a pressure difference obtained by subtracting an internal pressure of an artery from a pressure applied to a specific region is positive at the first timing, an obstacle obstructing a blood flow is generated in the artery. Further, a pulse wave is also generated due to collision of blood with the obstacle. With an increase in the pressure difference, the obstacle becomes stronger. As the obstacle becomes stronger, blood becomes likely to collide with the obstacle. As a result, the first timing is affected by the pressure difference. In other words, the first timing changes in a generation timing thereof in accordance with a magnitude of the pressure difference.

In this case, a largest (or approximately largest) pressure making no obstacle at the first timing is a diastolic blood pressure.

Further, the fourth timing is a timing of peak blood flow, which occurs due to pumping of blood by the heart, in a measurement region. At the fourth timing, a caliber of an artery is largest (or substantially largest). Further, an internal pressure of the artery is highest (or substantially highest) at the fourth timing. The fourth timing is affected by arterial compliance, changes in a blood flow, and the like. In other words, the fourth timing depends on a magnitude of the pressure difference.

Next, the pulse wave calculation unit 102 generates pulse wave information where the calculated period (hereinafter, expressed as a “pulse wave parameter”) and one pressure value of the plurality of pressure values are associated with each other.

In this case, a smallest (or approximately smallest) pressure generating an obstacle for stopping a blood flow at the fourth timing is a systolic blood pressure.

The pulse wave information is, for example, information where a pressure value and a pulse wave parameter are associated with each other as illustrated in FIG. 4. FIG. 4 is a diagram conceptually illustrating one example of the pulse wave information. The pulse wave information associates, for example, a pressure “70” and a pulse wave parameter “aa”. This represents that, when the pressure applied to a specific region is “70,” a value of the pulse wave parameter is “aa.”

It is not always necessary for the pulse wave information to associate a pressure in a certain period and a pulse wave parameter. The pulse wave information may be a parameter obtained for example, through regression analysis between the pressure and the pulse wave parameter. Further, the pulse wave information may be a value obtained in accordance with predetermined steps on the basis of the pressure or the pulse wave signal 2001 instead of a combination between the pulse wave parameter and the pressure. In other words, the pulse wave information is not limited to the above-described examples.

Next, the blood pressure estimation unit 103 estimates blood pressure (blood pressure value) for the pulse wave signal 2001 on the basis of the pulse wave information calculated by the pulse wave calculation unit 102 (step S203). The blood pressure is systolic blood pressure, diastolic blood pressure, or both thereof. The systolic blood pressure is measured when blood is pumped to an artery by contraction of the heart. On the other hand, the diastolic blood pressure is measured when blood is gently pumped to the artery while the heart dilates.

The blood pressure estimation unit 103 estimates blood pressure for the pulse wave signal 2001 on the basis of blood pressure information where pulse wave information and blood pressure value are previously associated with each other as exemplified in FIG. 5 and on the basis of the pulse wave information generated by the pulse wave calculation unit 102. FIG. 5 is a diagram conceptually illustrating one example of the blood pressure information. In this case, the blood pressure includes diastolic blood pressure and systolic blood pressure. Further, in the example of FIG. 5, the pulse wave information is information where a pressure at a certain timing and a pulse wave parameter calculated on the basis of a pulse wave signal are associated with each other. The blood pressure estimation device 101 may store the blood pressure information into itself, or may store the blood pressure information into an external storage device.

The blood pressure estimation unit 103 reads, from the blood pressure information, blood pressure associated with the received particular pulse wave information (i.e. information where a pulse wave parameter for the pulse wave signal 2001 and the pressure signal 2003 are associated). In other words, the blood pressure estimation unit 103 refers to the blood pressure information and thereby determines blood pressure associated with the received particular pulse wave information.

In the above-described example, the blood pressure estimation unit 103 searched pulse wave information coincident with particular pulse wave information in the blood pressure information, but may search similar (or coincident) pulse wave information by calculating a similarity degree between the particular pulse wave information and pulse wave information in the blood pressure information. The blood pressure estimation unit 103 may select pulse wave information having a similarity degree higher than a predetermined threshold and read a pressure associated with the selected pulse wave information. Further, blood pressure information associated with the particular pulse wave information may include blood pressure information for a plurality of subjects to be measured.

The blood pressure estimation unit 103 may select a piece of pulse wave information having a highest (or substantially highest) similarity degree and read blood pressure associated with the selected pulse wave information. The similarity degree is an extent of resembling two data and is, for example, calculated by a distance between the two data. In this case, the shorter the distance is, the higher the similarity degree is. The longer the distance is, the lower the similarity degree is. The similarity degree may be calculated as an angle between two vectors corresponding the two data respectively when two data is assumed to be represented as two vector.

Further, it is not always necessary for the blood pressure estimation unit 103 to calculate similarity degrees between all pieces of data of the pulse wave information in the blood pressure information and the particular pulse wave information, and a part of the pieces of data of the pulse wave information in the blood pressure information may be used. In this case, the maximum internal pressure of the cuff may not be controlled to become larger than a systolic blood pressure. For example, processing of increasing internal pressure of the cuff may be stopped when the similarity degree between the pulse wave information in the blood pressure information and particular pulse wave information generated under the increasing internal pressure of the cuff become larger than the predetermined threshold. Controlling internal pressure of the cuff as described above can alleviate a burden to a body through measurement.

Next, the blood pressure estimation device 101 estimates blood pressure (hereinafter, expressed as a “first blood pressure” for convenience of explanation) for the pulse wave information on the basis of the read blood pressure. When, for example, the number of the read blood pressures is one, the blood pressure estimation unit 103 estimates the read blood pressure as a first blood pressure. Further, when blood pressure read in accordance with a similarity degree is estimated, the blood pressure estimation unit 103 may estimate the blood pressure as a first blood pressure by, for example, calculating a weighted average value in accordance with the similarity degree.

The blood pressure information includes pulse wave information where blood pressure and pulse wave are associated and blood pressure. The blood pressure information may be information obtained through previous measurement for a plurality of subjects to be measured. The blood pressure information may be individual information for each subject to be measured.

Further, when there are a plurality of pieces of blood pressure information, the blood pressure estimation device 101 may synthesize new blood pressure information based on the plurality of pieces of blood pressure information. A method for the synthesis is, for example, a method of averaging a plurality of pieces of information or a method of summing pieces of data in a plurality of blood pressure information and then executing fitting a non-linear function to the results. In this case, blood pressure information synthesized by the blood pressure estimation device 101 may preferably include a combination at the same timing and parameters calculated using the same method. Further, target for the synthesized blood pressure information are preferably blood pressure information having equal to or larger than a predetermined reference value.

As described above, highly accurate blood pressure information having less noise can be obtained by synthesizing new blood pressure information on the basis of a plurality of pieces of blood pressure information.

In this case, the blood pressure estimation device 101 according to the present example embodiment reads, from blood pressure information, pulse wave information associated with particular pulse wave information or blood pressure associated with pulse wave information resembling (or matching) the particular pulse wave information. The blood pressure estimation device 101 estimates blood pressure for the particular pulse wave information on the basis of the read blood pressure. Therefore, the blood pressure estimation device 101 can estimate blood pressure while reducing an influence of the noise after reading blood pressure from blood pressure information even when a pulse wave or a pressure includes noise.

On the other hand, it is difficult for a common blood pressure measurement device to accurately measure blood pressure when a pulse wave to be measured includes noise, as described above.

In other words, in accordance with the blood pressure estimation device 101 according to the present example embodiment, blood pressure can be estimated with a high accuracy.

Further, the blood pressure estimation unit 103 may estimate systolic blood pressure by pressure at the largest (or substantially largest) difference signal in case of multiple pulse wave signals 2001.

Heart pumps much blood to an artery in a systolic period. In this case, since much blood flows in the artery at a time, pressure in the artery changes in accordance with a pumped blood amount. In other words, a pumped blood amount is larger in an upstream and a blood amount is smaller in a downstream. As a result, difference signals for pulse wave signals measured in the upstream and pulse wave signals measured in the downstream are greatly different. Therefore, the blood pressure estimation unit 103 can estimate systolic blood pressure by a pressure at the largest (or substantially largest) difference signal.

Further, the blood pressure estimation unit 103 may estimate diastolic blood pressure by a pressure in the case when a difference signal takes smaller value than a certain value in multiple pulse wave signals 2001.

The certain value is, for example, a value higher, by several percent to some tens percent, than an average value of difference signals in which no pressure is applied. Further, the certain value may be a value calculated on the basis of diastolic blood pressure measured in accordance with a method such as an oscillometric method or a Korotkoff method. The certain value is not limited to the above-described examples.

Heart gently pumps blood to an artery in a diastolic period. In this case, blood gently flows in the artery, and therefore, a pressure in the artery does not change to a large extent. As a result, a difference between a pulse wave signal measured in an upstream and a pulse wave signal measured in a downstream is small. Therefore, the blood pressure estimation unit 103 can estimate diastolic blood pressure by a pressure that is lower than a systolic blood pressure in case where a difference signal is smaller than a certain value.

In the above-described example, the difference signal may be a difference or a ratio. When the difference signal is a ratio, the blood pressure estimation unit 103 estimates blood pressure in accordance with a magnitude of the ratio. The difference signal may be a comparable index on a plurality of pulse wave signals, and is therefore not limited to the above-described example.

The blood pressure estimation device 101 estimates blood pressure on the basis of a difference signal. Therefore, even when, for example, multiple pulse wave signals include similar noise, the blood pressure estimation device 101 estimates blood pressure on the basis of a difference to reduce the noise. Therefore, the blood pressure estimation device 101 can reduce an influence of noise and estimate blood pressure with a high accuracy.

On the other hand, it is difficult for a common blood pressure estimation device to accurately measure blood pressure when a pulse wave to be measured includes noise, as described above.

In other words, according to the blood pressure estimation device 101 according to the present example embodiment, blood pressure can be estimated with a high accuracy.

In the above-described example, a range of the pressure signal 2003 included diastolic blood pressure and systolic blood pressure, but as exemplified in FIG. 6, it is not always necessary to include both blood pressures. FIG. 6 is a diagram illustrating one example of the pressure signal 2003 whose range does not include systolic blood pressure. The upper figure of FIG. 6 illustrates the pressure signal 2003. The lower figure of FIG. 6 illustrates the pulse wave signal 2001. The horizontal axis in FIG. 6 represents time, and indicates a later time toward the rightward side. The vertical axis in the upper figure of FIG. 6 represents a pressure, and the pressure increases toward the upper side. The vertical axis in the lower figure of FIG. 6 represents pulse wave, and represents that the pulse wave is stronger toward the upper side or the lower side and weaker toward zero. In the example illustrated in FIG. 6, the pulse wave signal 2001 is measured in a period until the pressure signal 200 is stopped.

Even when the range of the pressure signal 2003 does not include systolic blood pressure, the blood pressure estimation device 101 can estimate blood pressure on the basis of the pulse wave signal 2001 measured until the pressure signal 2003 is stopped.

The blood pressure estimation device 101 generates pulse wave information calculated by the pulse wave calculation unit 102, for example, on the basis of a received pulse wave signal 2001 and a pressure signal 2003. Then, the blood pressure estimation unit 103 compares the pulse wave information and pulse wave information (or a part of pulse wave information) in blood pressure information, selects similar (or coincident) pulse wave information, and reads blood pressure associated with the similar (or coincident) pulse wave information. The blood pressure estimation unit 103 estimates blood pressure for the received pulse wave signal on the basis of the read blood pressure.

The blood pressure estimation device 101 receives, for example, a pressure signal 2003 measured by a blood pressure measurement device 408 exemplified in FIG. 7 and a pulse wave signal 2001 measured by the blood pressure measurement device 408. FIG. 7 is a block diagram illustrating a configuration of the blood pressure measurement device 408 according to the first example embodiment.

The blood pressure measurement device 408 includes the cuff 401, the pulse wave measurement unit 402, the pressure measurement unit 407, the pressure control unit 404, the input unit 405, the display unit 406, and the blood pressure estimation device 101. FIG. 8 is a perspective view of the cuff 401 that is not attached. In FIG. 8, the blood pressure measurement device 408 includes a plurality of pulse wave measurement units but may include one pulse wave measurement unit. Further, in FIG. 8, the cuff 401 and the pulse wave measurement unit 402 are integrally formed, but the cuff 401 and the pulse wave measurement unit 402 may be connected via a pulse wave transmission unit (not depicted). The pulse wave transmission unit is, for example, a tube, and an internal pressure of the tube varies in accordance with a variation of an internal pressure of the cuff 401, whereby a pulse wave measured at a specific region is transmitted to the pulse wave measurement unit 402.

For convenience of explanation, it is assumed that a longer side direction is a direction where the cuff 401 is wound around a specific region. Further, it is assumed that a shorter side direction is a direction orthogonal (substantially orthogonal) to the longer side direction.

First, a subject to be measured winds the cuff 401 around a specific region such as an upper arm, a leg, a wrist, an ankle, or the like and measures blood pressure there as exemplified in FIG. 9. FIG. 9 is a diagram illustrating one example of a state where the cuff 401 is attached on a specific region. A subject to be measured winds the longer side direction around the specific region to attach the cuff 401. In this case, it is conceivable that an artery is parallel (or substantially parallel) to the shorter side direction.

The pulse wave measurement unit 402 is, for example, a vibration sensor that detects vibrations occurred in accordance with a pulse wave, a photoelectric pulse wave sensor that detects reflected light in which irradiated light is reflected or transmitted light in which irradiated light is transmitted. The pulse wave measurement unit 402 is, for example, an ultrasound sensor that detects reflection or transmission of irradiated ultrasound, an electric field sensor, a magnetic field sensor, or an impedance sensor.

Further, the pulse wave measurement unit 402 may be a pressure sensor. In a case of the pressure sensor, a pressure is decomposed into signals having cycles different from each other, for example, via Fourier transformation. When the pressure control unit 404 applies pressure or reduces pressure at a constant (or substantially constant) speed, a cycle for a pressure resulting from the pressure control unit 404 is long. Therefore, a pulse wave signal resulting from a pulse wave can be extracted by selecting a signal having a short cycle from the pressure.

The subject to be measured operates the input unit 405 and starts a measurement. The input unit 405 includes a measurement start button, a power button, a measurement stop button for canceling the measurement after the measurement start, and a left button and a right button used upon selecting an item displayed by the display unit 406 (each thereof being not depicted). The input unit 405 transmits an input signal received from a subject to be measured or the like to the blood pressure estimation device 101.

In response to the measurement start, the pressure control unit 404 controls an amount of gas (e.g. air), liquid, or both sealed in the cuff 401 while referring to internal pressure of the cuff 401 measured by the pressure measurement unit 407 and thereby controls pressure applied to a specific region. The pressure control unit 404 controls, for example, operations of a pump that sends the gas sealed in the cuff 401 and a valve in the cuff 401.

The cuff 401 may include a pressure bag (not depicted) in which gas and liquid are sealed. The cuff 401 accumulates fluid and the like in the pressure bag in accordance with control of the pressure control unit 404 and thereby applies a pressure to the specific region.

When there are a plurality of pulse wave measurement units, a plurality of pulse wave measurement units may be disposed so as to sandwich a pressurization center (or substantial pressurization center) of the shorter side direction of the cuff 401.

Then, while the pressure control unit 404 executes control for applying a pressure to the specific region, the pulse wave measurement unit 402 measures a pulse wave at the specific region.

The pulse wave measurement unit 402 transmits the measured pulse wave as a pulse wave signal 2001 to the blood pressure estimation device 101. The pressure measurement unit 407 transmits the measured pressure as a pressure signal to the blood pressure estimation device 101.

The pressure measurement unit 407 converts the measured pressure into a digital signal by discretization (analog digital conversion, or A/D conversion) of the measured pressure, and transmits the digital signal as a pressure signal 2003. In the same manner, the pulse wave measurement unit 402 converts the measured pulse wave into a digital signal, for example, by discretization of the measured pulse wave and transmits the digital signal as a pulse wave signal 2001.

A part of a pressure (or a pulse wave) may be extracted with a filter and the like for extracting particular frequency in A/D conversion. Further, pressure (or a pulse wave) may be amplified to a predetermined amplitude.

The blood pressure estimation device 101 estimates blood pressure via the above-described processing. In doing so, the blood pressure estimation device 101 may transmit a control signal that makes an instruction for a control content to the pressure control unit 404.

The display unit 406 displays the blood pressure calculated by the blood pressure estimation device 101. The display unit 406 is an LCD (Liquid Crystal Display), an OLED (Organic_light_emitting_diode), an electronic paper, or the like. The electronic paper can be realized in accordance with, for example, a microcapsule type, an electron powder fluid type, a cholesteric liquid crystal type, an electrophoretic type, an electrowetting type, or the like.

The blood pressure measurement device 408 includes the blood pressure estimation device 101 and can therefore estimate blood pressure with a high accuracy. In other words, according to the blood pressure measurement device 408 of the first example embodiment, blood pressure can be measured with a high accuracy.

The blood pressure measurement device 408 may include a manner in which the pulse wave measurement unit 402 executes transmission/reception of pulse wave information to/from the blood pressure estimation device 101 via a communication network (e.g. a wired communication network, a wireless communication network, or the like).

Further, the specific region may be an upper arm, a wrist, or the like. When the specific region is a wrist, the pulse wave measurement unit 402 may detect a pulse wave via a radial artery.

Further, the cuff 401 needs only to include a function for pressurizing an artery and may be a mechanical component, an artificial muscle or the like in which a pressure for pressurization is changed.

### SECOND EXAMPLE EMBODIMENT

Next, a second example embodiment of the present invention based on the above-described first example embodiment will be described.

In the following description, characteristic parts of the present example embodiment will be mainly described, and the same components as in the above-described first example embodiment are assigned with the same reference signs, whereby overlapping description will be omitted.

With reference to FIG. 10 and FIG. 11, a configuration of a blood pressure estimation device 901 according to the second example embodiment and processing executed by the blood pressure estimation device 901 will be described. FIG. 10 is a block diagram illustrating the configuration of the blood pressure estimation device 901 according to the second example embodiment of the present invention. FIG. 11 is a flowchart illustrating a flow of processing in the blood pressure estimation device 901 according to the second example embodiment.

The blood pressure estimation device 901 according to the second example embodiment includes a pulse wave calculation unit 902 and a blood pressure estimation unit 903.

The pulse wave calculation unit 902 calculates a timing on the basis of a pressure signal 2003 and a pulse wave signal 2001 and generates pulse wave information on the basis of the timing (step S901).

Hereinafter, with reference to FIG. 12, processing for calculating pulse wave information by the pulse wave calculation unit 902 will be described. FIG. 12 is a cross-sectional view schematically illustrating a pressure signal 2003 and a specific region where a pulse wave signal is measured.

For convenience of explanation, hereinafter, a value obtained by subtracting an internal pressure of an artery at measurement region of a pulse wave from the pressure signal 2003 will be expressed as a “pressure difference.”

First, the cuff 401 applies pressure to an artery wall 1103 via a skin 1101 and a subcutaneous tissue 1102. When the pressure applied by the cuff 401 is sufficiently high, an obstacle 1105 obstructing a blood flow 1104 is formed in the artery.

When the pressure signal 2003 is lower than a diastolic blood pressure (a state “a” illustrated in FIG. 12), the pressure difference is equal to or smaller than zero. Therefore, the artery wall 1103 is not deformed by the pressure in the pressure signal 2003. In this case, in accordance with the blood flow 1104 flowing in the artery, an internal pressure of the artery is changed, and therefore, an internal diameter of the artery is changed in accordance with the change of the internal pressure of the artery. Therefore, the pulse wave signal is a pulse wave in accordance with the internal pressure of the artery without an influence of the pressure signal 2003.

On the other hand, when the pressure signal 2003 is higher than diastolic blood pressure and the pressure difference has a positive value (a state b illustrated in FIG. 12), the artery is subjected to a pressure represented by the pressure signal 2003, and thereby an obstacle 1105 obstructing the blood flow 1104 is formed in the artery wall 1103. In this case, in the artery wall 1103, not only a deformation caused by the pressure signal 2003 but also a deformation of a blood flow direction due to collision of the blood flow 1104 with the formed obstacle 1105 are generated. Further, with an increase in the pressure difference, the artery wall 1103 is contracted and arterial compliance is decreased, and therefore, a speed of deformation in the blood flow direction is changed. Further, with an increase in the pressure difference, a large obstacle 1105 is likely to be formed, and in addition, it becomes difficult for the artery wall 1103 to return to a normal state. Therefore, when a shape of a pulse wave upon applying a pressure and a shape of a pulse wave upon applying no pressure are compared, with an increase in the pressure difference, the shape of the pulse wave is greatly changed.

When the pressure signal 2003 is higher than a systolic blood pressure, the obstacle 1105 occludes the blood flow 1104 in the artery. In this case, in the artery wall 1103, a deformation of a blood flow direction is mainly generated due to collision of the blood flow 1104 with the obstacle 1105. Even when the pressure signal 2003 is higher, a situation in which the obstacle 1105 occludes a blood flow in the artery is kept unchanged. Therefore, when the pressure signal 2003 is higher than the systolic blood pressure, a deformation of the blood flow direction is not significantly changed in the artery wall 1103. In other words, even in a case of a higher pressure, a shape of the pulse wave signal 2001 is not substantially changed from a shape of the pulse wave signal 2001 in the case of systolic blood pressure.

As a result, there is a relation, as illustrated in FIG. 13, between a magnitude of a change between a shape (hereinafter, referred to “first shape”) of a pulse wave upon applying no pressure and a shape (hereinafter, referred to “second shape”) of the pulse wave signal 2001 upon applying a pressure and the pressure signal 2003. FIG. 13 is a diagram conceptually illustrating one example of a relation between the pressure signal 2003 and a magnitude of change from the first shape to the second shape. The horizontal axis of the FIG. 13 shows a pressure and represents that the pressure is higher toward the right side. The vertical axis of the FIG. 13 shows the magnitude of change from the first shape to the second shape and represents that the change is larger toward the upper side.

When the pressure signal 2003 is equal to or smaller than diastolic blood pressure (“DBP” in FIG. 13), a magnitude of a change from the first shape to the second shape is small and is constant (or substantially constant) regardless of the pressure signal 2003. When the pressure signal 2003 lies somewhere between diastolic blood pressure and systolic blood pressure, with an increase in the pressure signal 2003, the magnitude of a change from the first shape to the second shape is large. Further, when the pressure signal 2003 is equal to or larger than systolic blood pressure, the magnitude of a change from the first shape to the second shape is large and is constant (or substantially constant) regardless of the pressure signal 2003.

With reference to FIG. 14, an example of processing for calculating a timing in the pulse wave calculation unit 902 will be described. FIG. 14 is a diagram conceptually illustrating one example of processing for calculating a timing.

The timing is, for example, a point of time when a derivation signal obtained by an n-th order differentiation (n is an integer equal to or larger than 0) of the pulse wave signal with respect to time is zero if a pulse wave signal (i.e. the pulse wave signal 2001 in this example) and the pulse wave signal are continuous. Alternatively, the timing is a point of time when a derivation signal as a result obtained by applying, for example, an n-stage difference (n is an integer equal to or larger than 0) to the pulse wave signal with respect to time is the closest to zero if the pulse wave signal is a discrete signal.

The horizontal axis of FIG. 14 represents time and represents that time is later toward the right side. The vertical axis of FIG. 14 represents a signal and represents that the signal is stronger toward the upper side. Four curves in FIG. 14 each are, in order from the top, a pressure signal 2003, a pulse wave signal 2001, a derivation signal (hereinafter, expressed as a “first derivation signal”) as a result obtained by primarily differentiating the pulse wave signal 2001 with respect to time, and a derivation signal (hereinafter, expressed as a “second derivation signal”) as a result obtained by secondarily differentiating the pulse wave signal 2001 with respect to time.

The pulse wave calculation unit 902 calculates a timing when the pulse wave signal 2001, the first derivation signal, or the second derivation signal has a certain value.

The pulse wave calculation unit 902 calculates, for example, a first timing 81 when the pulse wave signal is smallest (or substantially smallest) in one heartbeat (i.e. one cycle). In other words, a pulse wave signal starts rising at the first timing 81.

The pulse wave calculation unit 902 estimates the first timing 81, for example, as a timing when an inclination of the pulse wave signal 2001 is equal to or larger than a predetermined inclination. In other words, the pulse wave calculation unit 902 may estimate the first timing 81 as a timing when the first derivation signal is equal to or larger than a first threshold. In this case, the first threshold is a value equal to or larger than zero.

Further, the pulse wave calculation unit 902 may calculate a timing when a second derivation signal becomes equal or larger than a second threshold, if there are a plurality of timings when the first derivation signal is equal to or larger than the first threshold in one cycle. This processing enables the pulse wave calculation unit 902 to calculate the first timing 81 more accurately.

The pulse wave calculation unit 902 calculates, for example, a second timing when an inclination of the pulse wave signal 2001 increases in one cycle.

An obstacle 1105 in an artery disappears at a second timing 82. The obstacle 1105 is formed at the first timing 81 and thereafter a pressure difference becomes negative after pumping of blood by heart, whereby the obstacle 1105 disappears. When the obstacle 1105 disappears, a deformation in a direction vertical to a blood flow 1104 increases after pumping of blood by heart, and therefore, a changing speed of the pulse wave signal 2001 increases.

The pulse wave calculation unit 902 may estimate the second timing 82 as a timing when the second derivation signal exceeds the second threshold in one cycle. The pulse wave calculation unit 902 may estimate the second timing 82 as a timing when the second derivation signal becomes local maximum (or substantially local maximum) in one cycle.

For example, “substantially local maximum” can be defined as a value that is within a certain range from the local maximum. The certain range may be a value calculated on the basis of a fact in which a magnitude of an inclination (determined by calculating a differential, difference, or the like) of a target for which a maximum value is calculated is less than a predetermined value. The certain range is not limited to the above-described example.

When the second derivation signal includes a plurality of local maximum values in one cycle, the pulse wave calculation unit 902 may refer to a third derivation signal obtained by cubic differentiation of a pulse wave signal with respect to time, a fourth derivation signal obtained by quartic differentiation of a pulse wave signal with respect to time, or the like and calculate the second timing 82. In other words, the method for calculating the second timing 82 is not limited to the above-described example.

The pulse wave calculation unit 902 estimates, for example, a third timing 83 as a timing when the first derivation signal becomes maximum (or in a maximum vicinity) in one cycle. In other words, a dilation speed of an artery at the third timing 83 is largest (or substantially largest).

A pressure difference becomes negative and thereafter the artery further dilates depending on pumping of blood by heart. Unless the artery does not rupture, the dilation of the artery stops soon. Therefore, the dilation speed of the artery becomes largest (or substantially largest). In other words, this timing is the third timing 83.

At the third timing 83, arterial compliance decreases due to a pressure of the pressure signal 2003. The third timing 83 is affected by a factor such as a decrease in a blood flow due to an obstacle 1105 having been formed while the pressure difference is positive. In other words, the third timing 83 changes in accordance with the pressure difference.

The pulse wave calculation unit 902 calculates, for example, a fourth timing 84 when a difference is largest (or substantially largest). The pulse wave calculation unit 902 may calculate the fourth timing 84, on the basis of, for example, a timing when the first derivation signal becomes 0 (or substantially 0) or a timing when the second derivation timing is convex downward. In other words, the method for calculating the fourth timing 84 is not limited to the above-described examples.

The pulse wave calculation unit 902 calculates, for example, a fifth timing 85 when the first derivation signal is smallest (or substantially smallest) in one cycle. In other words, at the fifth timing 85, a contraction speed of an artery is largest (or substantially largest).

When a peak of pumping of blood by heart is passed, an internal pressure of an artery is decreased. The artery contracts depending on a decrease of the internal pressure of the artery. The contraction speed of the artery becomes largest (or substantially largest) soon.

The fifth timing 85 is affected by arterial compliance or the like in the same manner as the third timing 83. In other words, the fifth timing 85 is determined in accordance with a pressure difference or the like.

The pulse wave calculation unit 902 calculates, for example, a sixth timing 86 when the second derivation signal exceeds a predetermined value in one cycle. Alternatively, the pulse wave calculation unit 902 may estimate the sixth timing 86 as a timing when the second derivation signal is local maximum (or substantially local maximum) in one cycle.

In the sixth timing, an obstacle 1105 is formed in an artery. A peak of pumping of blood by heart has been passed, and therefore, an internal pressure of the artery decreases. When a pressure difference becomes negative, the obstacle 1105 is generated in the artery. The obstacle 1105 is generated, and thereby a changing speed of a pulse wave signal is unlikely to be affected by the internal pressure of the artery. As a result, a decreasing speed of the changing speed of the pulse wave signal becomes rapidly small.

When there are a plurality of timings when the second derivation signal is local maximum (or substantially local maximum) in one cycle, the pulse wave calculation unit 902 may estimate the sixth timing 86 as a timing when the third derivation signal is local maximum (or substantially local maximum) or a timing when the fourth derivation signal is local maximum (or substantially local maximum). In other words, the method for calculating the sixth timing 86 is not limited to the above-described examples.

The first timing 81 to the sixth timing 86 can be calculated on the basis of a pressure signal, a derivation signal, or a pulse wave signal, and therefore, the calculation method is not limited to the above-described examples.

An example of processing in which the pulse wave calculation unit 902 calculates pulse wave information on the basis of multiple pulse wave signals will be described.

The pulse wave calculation unit 902 calculates, for example, a difference between two timings in the first timing 81 to the sixth timing 86 and thereby calculates a period between the two timings. The pulse wave calculation unit 902 need not always calculate a period in one heartbeat, and may estimate the period as a difference between two timings over multiple heartbeats. When calculating the difference between two timings over multiple heartbeats, the pulse wave calculation unit 902 may calculate a difference between timings in multiple heartbeats by using one kind of timing.

Further, the method for calculating a period may be a method for calculating a difference between the above-described timing and a reference timing. In this case, the blood pressure estimation device 901 calculates the reference timing on the basis of, for example, a waveform output by an electrocardiograph.

The reference timing is a timing synchronizing with a cycle of the heartbeats and is not influenced by the obstacle 1105. The reference timing is, for example, a timing representing a characteristic such as an R wave, a Q wave, an S wave, a P wave, or a T wave in an electrocardiogram.

The reference timing is not subjected to an influence resulting from the obstacle 1105, and therefore, the pulse wave calculation unit 902 can calculate a period with a higher degree of accuracy.

Further, the pulse wave calculation unit 902 may normalize the above-described period. A method for the normalization is, for example, a method for calculating a ratio between a determined period and a heartbeat cycle (e.g. a peak interval of pulse waves, an R-R interval of an electrocardiogram, or the like), a method for determining a ratio between a plurality of periods calculated by combining different characteristic points, or the like. The method for the normalization is not limited to the above-described examples. The normalization makes it possible to correct an influence produced by different heartbeat cycles in a pulse wave signal, and therefore the pulse wave calculation unit 902 calculates a more accurate period.

Next, a method in which the pulse wave calculation unit 902 calculates a pressure in a period between a particular first timing and a particular second timing will be described.

The pulse wave calculation unit 902 designates, as a pressure, a pressure value of a pressure signal 2003 at the particular first timing or a pressure value of a pressure signal 2003 at the particular second timing. Further, the pulse wave calculation unit 902 may extrapolate, for example, the pressure value of the pressure signal 2003 at the particular first timing and calculate a pressure in a different heartbeat. In other words, the method in which the pulse wave calculation unit 902 calculates a pressure is not limited to the above-described example.

With reference to FIG. 15, characteristics included in pulse wave information will be described. FIG. 15 is a diagram conceptually illustrating characteristics included in pulse wave information. The horizontal axis of FIG. 15 represents pressure, and represents that the pressure is higher toward the right side. The vertical axis of FIG. 15 represents a pulse wave parameter, and represents that a period is longer toward the upper side. Five curves shown in FIG. 15 represent a relation between pressure and a period during between the particular first timing defined by the fourth timing 84 and the particular second timing different from the first timing (i.e. the first timing 81 to the third timing 83, the fifth timing 85, or the sixth timing 86). In this example, the pressure is a value of the pressure signal 2003 at the fourth timing 84.

It is assumed that a first curve 1581 is a curve representing a relation between the first timing 81 and the fourth timing 84. It is assumed that a second curve 1582 is a curve representing a relation between the second timing 82 and the fourth timing 84. It is assumed that a third curve 1583 is a curve representing a relation between the third timing 83 and the fourth timing 84. It is assumed that a fifth curve 1585 is a curve representing a relation between the fifth timing 85 and the fourth timing 84. It is assumed that a sixth curve 1586 is a curve representing a relation between the sixth timing 86 and the fourth timing 84.

The pressure in the five curves shown in FIG. 15 is normalized by setting diastolic blood pressure to 0 and setting systolic blood pressure to 100. In this example, the diastolic blood pressure and the systolic blood pressure each are a value measure according to an auscultatory method.

The curve representing a relation between a period and pressure includes characteristics as exemplified in FIG. 15. The five curves are different from each other depending on the particular second timing. The reason is that the particular first timing and the particular second timing are changed in accordance with various factors such as an artery as described above and are not changed uniformly with respect to the pressure.

When, for example, the pressure lies somewhere between a diastolic blood pressure and a systolic blood pressure, the first timing 81, the fourth timing 84, and the fifth timing 85 greatly change up and down. On the other hand, when the pressure does not fall within the above-described range, the first timing 81, the fourth timing 84, and the fifth timing 85 do not change to a large extent.

The blood pressure estimation unit 103 estimates blood pressure on the basis of this property. Further, the blood pressure estimation unit 103 may read blood pressure associated with pulse wave information from the blood pressure information and estimate the read blood pressure as blood pressure for the pulse wave information.

The blood pressure estimation device 901 estimates blood pressure on the basis of a pulse wave parameter representing a difference between the above-described timings. Therefore, even when a pulse wave signal includes noise, the noise can be eliminated by calculating the difference. As a result, by using the blood pressure estimation device 901 according to the present example embodiment, blood pressure can be estimated with a high accuracy.

On the other hand, a common blood pressure measurement device estimates blood pressure on the basis of a pulse wave signal, as described above. Therefore, when a pulse wave signal includes noise, it is difficult for the blood pressure measurement device to eliminate the noise and is therefore unable to estimate blood pressure accurately.

In the above-described example, as illustrated in FIG. 15, there is a positive correlation between a period and pressure. Even when a period and pressure has a negative correlation in accordance with a combination of the particular first timing and the particular second timing, the blood pressure estimation device 901 can estimate blood pressure in the same manner as the above-described processing.

With reference to examples illustrated in FIG. 16 and FIG. 17, processing of the blood pressure estimation unit 903 will be described. FIG. 16 is a diagram conceptually illustrating one example of a relevance between a pressure signal 2003 and a pulse wave parameter under increasing pressure. FIG. 17 is a diagram conceptually illustrating an example in which a curve representing a relevance between the pressure signal 2003 and the pulse wave parameter is estimated.

The horizontal axis in FIG. 16 represents pressure and represents that the pressure is higher toward the right side. The vertical axis in FIG. 16 represents a value of a pulse wave parameter and represents that the pulse wave parameter has a larger value toward the upper side. The horizontal axis in FIG. 17 represents pressure and represents that the pressure is higher toward the right side. The vertical axis in FIG. 17 represents a value of a pulse wave parameter and represents that the pulse wave parameter has a larger value toward the upper side.

As exemplified in FIG. 16, pulse wave information need not be discrete such as information where a pressure and a period are associated with each other. The pulse wave information may be, for example, a curve where pressure and a pulse wave parameter are associated or a parameter representing the curve. Further, the pulse wave information may be, as exemplified in FIG. 17, a curve in which a value of a pulse wave parameter is interpolated via extrapolation or a function on pressure and a period as parameters.

Further, the pulse wave information may be normalized on the basis of blood pressure or the like.

As illustrated in FIG. 17, for example, a method for extrapolating a curve includes a method for fitting (applying) pulse wave information to a predetermined function with a least-square method and a method for fitting a curve in accordance with pattern matching.

The blood pressure estimation unit 903 fits a curve to pulse wave information in which values are discretely provided. As a result, the pulse wave information is represented as the curve. The curve rises and falls, as described above, in accordance with a case in which pressure is lower than diastolic blood pressure, a case in which pressure lies somewhere between diastolic blood pressure and systolic blood pressure, and a case in which pressure is higher than systolic blood pressure. Therefore, the blood pressure estimation unit 903 can estimate diastolic blood pressure and systolic blood pressure on the basis of a rise and fall of the fitted curve.

As curve fitting for pulse wave information is more accurate, estimation of blood pressure is more accurate. When, for example, pressure in pulse wave information includes a value between systolic blood pressure and diastolic blood pressure, the blood pressure estimation unit 903 fits a curve to the pulse wave information with a high accuracy. Therefore, the blood pressure estimation unit 903 estimates blood pressure with a high accuracy.

In addition, when pressure in pulse wave information further includes a value equal to or larger than systolic blood pressure or a value equal to or smaller than diastolic blood pressure, the blood pressure estimation unit 903 fits a curve to the pulse wave information with a higher degree of accuracy. Therefore, the blood pressure estimation unit 903 estimates blood pressure with a higher degree of accuracy.

It is not always necessary for the blood pressure estimation device 901 to calculate pulse wave information on the basis of a pulse wave signal 2001 measured under pressure including pulse wave information including systolic blood pressure and diastolic blood pressure. In this case, the blood pressure estimation device 901 calculates particular pulse wave information on the basis of a pressure signal 2003 that does not always include systolic blood pressure and diastolic blood pressure, and on the basis of a pulse wave signal 2001 measured under pressure of the pressure signal 2003. The blood pressure estimation device 901 estimates, as first blood pressure, blood pressure associated with pulse wave information similar to (or coincident with) the particular pulse wave information in blood pressure information.

When, for example, a similarity degree between the particular pulse wave information and pulse wave information in the blood pressure information exceeds a predetermined threshold, the blood pressure estimation device 901 may estimate blood pressure associated with the pulse wave information as the first blood pressure.

In this case, a blood pressure measurement device (not depicted) including the blood pressure estimation device 901 may terminate processing for measuring blood pressure such as processing for stopping pressurization or processing for depressurization in accordance with a fact that it becomes possible for the blood pressure estimation device 901 to estimate the first blood pressure.

An upper limit of pressure is not specifically limited and may be set in a range of pressure lower than systolic blood pressure to the extent of physical burden that a subject to be measured does not feel under the pressure.

Further, the blood pressure estimation unit 903 may estimate blood pressure index value different from diastolic blood pressure or systolic blood pressure without fitting a curve. The blood pressure index value is, for example, an average blood pressure value. In this case, the blood pressure estimation unit 903 estimates pressure at a timing when an envelope for amplitudes in a pulse wave signal is largest (or approximately largest), as in an oscillometric method as the average blood pressure value.

As described above, the blood pressure estimation device 901 may estimate blood pressure on the basis of pulse wave information. Even when the pulse wave information is discrete information, the blood pressure estimation device 901 determines a curve fitting to the pulse wave information and thereby estimates blood pressure for a pulse wave signal. Therefore, a blood pressure measurement device including the blood pressure estimation device 901 according to the present example embodiment can shorten a time for imposing a burden to a subject to be measured and further alleviate a physical burden accompanied with measurement.

Further, the blood pressure estimation device 901 calculates a pulse wave parameter representing a difference between the above-described timings even when pulse wave information includes noise. Since the noise decreases through calculation of the pulse wave parameter, by using the blood pressure estimation device 901 according to the present example embodiment, blood pressure can be estimated with a high accuracy without an influence of noise occurred by, for example, body movements or the like.

Hereinafter, noise reduction by calculating a difference signal will be described.

Movements in a subject to be measured, vibrations from the outside, noise from a surrounding area, and the like occurs noise signals and the noise signals are added into pulse wave information.

For convenience of explanation, measured signals including noise signals are denoted by S1 and S2, and pulse wave signals related to the subject to be measured are denoted by P1 and P2.

In this case, the measurement signals and the pulse wave signals have the relationships expressed by Equation 1 and Equation 2 below. Specifically,

S1=P1×a1+b1 (Equation 1)

S2=P2×a2+b2 (Equation 2)

(where a1 and a2 respectively denote multiplication noise for the pulse wave signal S1 and multiplication noise for the pulse wave signal S2, and b1 and b2 respectively denote addition noise for the pulse wave signal S1 and addition noise for the pulse wave signal S2).

Here, k is defined according to Equation 3 below. Specifically,

k=b1/b2 (Equation 3).

Equation 4 below is established on the basis of Equation 1, Equation 2, and Equation 3 described above. Specifically,

S1−k×S2=P1×a1−P2×k×a2 (Equation 4).

When a1 and a2 are sufficiently close to one (i.e., each multiplication noise is sufficiently small), or when a characteristic value that is not affected by any multiplication noise is extracted, a1 and a2 can be ignored, consequently reducing noise.

Here, m is defined according to Equation 5 below. Specifically,

m=a1/a2 (Equation 5).

Equation 6 below is established on the basis of Equation 1, Equation 2, and Equation 5 described above. Specifically,

S1/m/S2=(P1+b1/a1)/(P2+k×b2/a1) (Equation 6)

When b1 and b2 are sufficiently small with respect to a1 and a2, respectively, or when a characteristic value that is not affected by any addition noise is extracted, a1 and a2 can be ignored, consequently reducing noise.

Multiplication noise and addition noise are non-independently added to multiple pulse wave signals measured by multiple pulse wave measurement units located at positions close to each other. In this case, even when the values k and m are not determined, noise signal components can be reduced by calculating the difference.

Hence, the blood pressure estimation device 901 according to the second example embodiment can estimate blood pressure with a high accuracy.

When a blood pressure measurement device 1007 including the blood pressure estimation device 901 measures three pulse waves as illustrated in FIG. 18, the blood pressure estimation device 901 can also estimate blood pressure as the above-described example. FIG. 18 is a diagram schematically illustrating a positional relationship between a cuff 1005 and three pulse wave measurement units.

For convenience of explanation, FIG. 18 shows a specific region and a blood flow and the like in the specific region, too. However, the blood pressure measurement device 1007 does not include the specific region and the blood flow and the like in the specific region.

The blood pressure measurement device 1007 includes a pulse wave measurement unit 1001, a pulse wave measurement unit 1002, a pulse wave measurement unit 1003, and the cuff 1005. The cuff 1005 may include a pressure bag 1006. At least two pulse wave measurement units of the pulse wave measurement unit 1001, the pulse wave measurement unit 1002, and the pulse wave measurement unit 1003 are located at positions so that pressurization center (or substantially center) in the shorter-side direction of the pressure application in the cuff 1005 is located between the pulse wave measurement units.

Each of the pulse wave measurement unit 1001, the pulse wave measurement unit 1002, and the pulse wave measurement unit 1003 measures a pulse wave at the specific region.

Here, for convenience of explanation, measurement signals including noise are denoted by S1, S2, and S3, and pulse signals are denoted by P1, P2, and P3.

In this case, the measurement signals and the pulse wave signals have the relationships expressed by Equation 7 to Equation 9 below. Specifically,

S1=P1×a1+b1 (Equation 7)

S2=P2×a2+b2 (Equation 8)

S3=P3×a3+b3 (Equation 9)

(where a1, a2, and a3 each denote multiplication noise for the corresponding pulse wave signal, and b1, b2, and b3 each denote addition noise for the corresponding pulse wave signal).

Here, k1 is defined according to Equation 10 below, and k2 is defined according to Equation 11 below. Specifically,

k1=b1/b2 (Equation 10)

k2=b1/b3 (Equation 11)

By calculating the difference between Equation 7 and Equation 8 and the difference between Equation 7 and Equation 9, Equation 12 and Equation 13 below are established. Specifically,

S1−k1×S2=P1×a1−P2×k1×a2 (Equation 12)

S1−k2×S3=P1×a1−P3×k2×a3 (Equation 13)

By calculating (Equation 12)/(Equation 13), Equation 14 below is established. Specifically,

(S1−k1×S2)/(S1−k2×S3)=(P1−P2×k1×a2/a1)/(P1−P3×k2×a3/a1) (Equation 14)

Equation 14 indicates that, when a1 is sufficiently close to a2 and a3 after the influences of the addition noises b1, b2, and b3 are cancelled, the influences of the multiplication noises can be ignored. This indicates that noise can be reduced.

Further, the noise signals (a1, a2, a3, b1, b2, and b3) are non-independently added to multiple pulse signals measured by multiple pulse wave measurement units located at positions close to each other. Accordingly, Equation 14 indicates that the influences of these noises can be reduced by calculating the difference even when the values k1 and k2 are not determined.

Hence, the blood pressure estimation device 901 according to the second example embodiment can reduce the influences of noise by estimating blood pressure on the basis of three or more pulse wave signals as described above.

Further, as illustrated in FIG. 19, when a blood pressure measurement device 1008 including the blood pressure estimation device 901 also measures four pulse waves, the blood pressure measurement device can estimate blood pressure in the same manner as in the above-described example. FIG. 19 is a diagram conceptually illustrating a position relation between a cuff 1005 and four pulse wave measurement units.

For convenience of explanation, FIG. 19 also illustrates a specific region and a blood flow and the like in the specific region. However, the blood pressure measurement device 1008 does not include the specific region or the blood flow and the like in the specific region.

The blood pressure measurement device 1008 includes a pulse wave measurement unit 1001, a pulse wave measurement unit 1002, a pulse wave measurement unit 1003, and a pulse wave measurement unit 1004, and a cuff 1005. The cuff 1005 may include a pressure bag 1006. At least two pulse wave measurement units of the pulse wave measurement unit 1001, the pulse wave measurement unit 1002, the pulse wave measurement unit 1003, and the pulse wave measurement unit 1004 are located at positions that sandwich a pressurization center (or substantially a pressurization center) of a shorter side direction in the cuff 1005.

The pulse wave measurement unit 1001, the pulse wave measurement unit 1002, the pulse wave measurement unit 1003, and the pulse wave measurement unit 1004 each measure a pulse wave in a specific region.

The blood pressure estimation device 901 estimates blood pressure in manner similar to the above-described processing, on the basis of the pulse wave measurement unit 1001, the pulse wave measurement unit 1002, the pulse wave measurement unit 1003, and the pulse wave measurement unit 1004.

Therefore, the blood pressure estimation device 901 according to the second example embodiment estimates blood pressure on the basis of four or more pulse wave signals and can thereby reduce an influence of noise on the basis of reasons similar to the above-described reason.

### THIRD EXAMPLE EMBODIMENT

Next, a third example embodiment of the present invention based on the above-described first example embodiment will be described.

In the following description, characteristic portions according to the present example embodiment will be mainly described, and the same components as in the above-described first example embodiment are assigned with the same reference signs, whereby overlapping description will be omitted.

With reference to FIG. 20 and FIG. 21, a configuration of a blood pressure measurement device 1201 according to the third example embodiment and processing executed by the blood pressure measurement device 1201 will be described. FIG. 20 is a block diagram illustrating the configuration of the blood pressure measurement device 1201 according to the third example embodiment of the present invention. FIG. 21 is a flowchart illustrating a flow of processing in the blood pressure measurement device 1201 according to the third example embodiment.

The blood pressure measurement device 1201 includes a cuff 401, a pulse wave measurement unit 402, a pressure measurement unit 407, a pressure control unit 1203, an input unit 405, a display unit 406, and a blood pressure estimation device 1202.

First, the pressure control unit 1203 executes control for applying an internal pressure of the cuff 401 in accordance with a start of measurement (step S1301). The pressure measurement unit 407 measures the internal pressure of the cuff 401 in a process of pressurization and transmits the measured pressure to the blood pressure estimation device 1202 as a pressure signal 2003 (step S1302). Further, the pulse wave measurement unit 402 measures a pulse wave at a specific region and transmits the measured pulse wave to the blood pressure estimation device 1202 as a pulse wave signal (step S1302)

The blood pressure estimation device 1202 receives the pressure signal 2003 and the pulse wave signal and calculates timings and a period (a pulse wave parameter) between a plurality of the timings on the basis of the received pressure signal 2003 and pulse wave signal (step S1303). The blood pressure estimation device 1202 generate, as particular pulse wave information, pulse wave information in which the pressure in the period and the pulse wave parameter are associated (step S1304).

Next, the blood pressure estimation device 1202 reads a pressure associated with the particular pulse wave information and outputs the blood pressure as blood pressure for the pulse wave signal (step S1305). Thereafter, the blood pressure measurement device 1201 reduces the internal pressure of the cuff 401 (step S1306).

In the above-described example, the blood pressure measurement device 1201 measured a pulse wave after an internal pressure was applied to the cuff but may measure a pulse wave in a process of decreasing internal pressure of the cuff after increasing the pressure to become equal or larger than systolic blood pressure.

Further, it is not always necessary for the blood pressure estimation device 1202 to calculate all pulse wave parameters when another pulse wave parameter can be estimated based on a calculated pulse wave parameter. In this case, it is not always necessary for the blood pressure measurement device 1201 to apply the internal pressure close to systolic blood pressure. Therefore, the blood pressure measurement device 1201 according to the present example embodiment can further shorten a measurement period and alleviate a burden on a subject to be measured because systolic blood pressure can be determined under lower pressure than common blood pressure measurement devices.

Further, the blood pressure measurement device 1201 according to the third example embodiment includes components similar to those in the first example embodiments, and therefore, effects similar to those in the first example embodiment can be obtained from the third example embodiment. In other words, the blood pressure measurement device 1201 according to the third example embodiment can measure blood pressure with a high accuracy.

### FOURTH EXAMPLE EMBODIMENT

Next, a fourth example embodiment of the present invention based on the above-described third example embodiment will be described.

In the following description, characteristic portions according to the present example embodiment will be mainly described and the same components as in the above-described third example embodiment are assigned with the same reference signs, whereby overlapping description will be omitted.

With reference to FIG. 22, a configuration of a blood pressure measurement device 2501 according to the fourth example embodiment and processing executed by the blood pressure measurement device 2501 will be described. FIG. 22 is a block diagram illustrating the configuration of the blood pressure measurement device 2501 according to the fourth example embodiment of the present invention.

The blood pressure measurement device 2501 further includes a determination unit 2502 and a correction unit 2503 in addition to the configuration of the third example embodiment.

The determination unit 2502 determines whether or not parameters for a state of a subject to be measured, parameters for surrounding environments and the like affect blood pressure to be estimated.

For example, the determination unit 2502 determines that the parameters affect blood pressure when, for example, a fitting curve for pulse wave information is changed depending on the parameters.

The parameters for a state of a subject to be measured include, for example, a parameter for behavior information (e.g. a recumbent position, a standing position, and a sitting position) on a body position, an activity amount or the like, or a parameter for vital information on the body temperature or a heartbeat number. Further, the parameters for a surrounding environment include, for example, a parameter for an atmosphere temperature, an atmosphere temperature near the body surface, or a temperature.

The parameters for a state of a subject to be measured include, for example, a value calculated in such a manner that a dynamic sensor such as an acceleration sensor, an angular speed sensor, or a clinometer is attached to a subject to be measured and a common behavior analysis algorithm is applied to a value output by the attached sensor. Further, the parameters for a surrounding environment include a value output by a temperature sensor placed in a circumference of a subject to be measured.

When the determination unit 2502 determines that the parameters affect blood pressure, the correction unit 2503 selects blood pressure information on the basis of the parameters (hereinafter, expressed as a “first parameter” for convenience of explanation) and pulse wave information. In this case, the blood pressure information associates pulse wave information, blood pressure information, and the parameters with each other. The correction unit 2503 reads, for example, pulse wave information associated with the parameters (i.e. the first parameter) representing behavior information from the blood pressure information. Thereafter, a blood pressure estimation device 1402 estimates blood pressure on the basis of the pulse wave information read by the correction unit 2503.

The correction unit 2503 may correct blood pressure information selected according to the pulse wave information on the basis of the parameters. When, for example, the parameters and blood pressure are highly correlated, the correction unit 2503 corrects the blood pressure estimated by the blood pressure estimation device 1402 on the basis of the correlation. The correction unit 2503 estimates, for example, blood pressure (expressed as a “first blood pressure”) on the basis of the correlation between the parameters and blood pressure and executes processing and the like for calculating a weighted average of the estimated first blood pressure and the blood pressure estimated by the blood pressure estimation device 1402 to correct the blood pressure.

The blood pressure measurement device 2501 according to the fourth example embodiment includes components similar to those in the third example embodiment, and therefore, effects similar to those in the third example embodiment can be obtained from the fourth example embodiment. In other words, the blood pressure measurement device 2501 according to the fourth example embodiment, blood pressure can be estimated with a high accuracy.

Further, the correction unit 2503 corrects blood pressure on the basis of parameters and the like representing behavior information and vital information. As a result, the blood pressure measurement device 2501 can measure blood pressure with a high accuracy regardless of a measurement environment.

An aspect may be employed in which while the blood pressure measurement device 2501 measures blood pressure when the determination unit 2502 determines that blood pressure is not affected, the blood pressure measurement device 2501 does not measure blood pressure when the determination unit 2502 determines that blood pressure is affected. Alternatively, an aspect may be employed in which when the determination unit 2502 determines that blood pressure is affected, the blood pressure measurement device 2501 promotes re-measurement or displays a message for requiring a subject to be measured to adjust his/her posture. Alternatively, an aspect may be employed in which the blood pressure measurement device 2501 does not start measurement until the determination unit 2502 determines that blood pressure is not affected.

### FIFTH EXAMPLE EMBODIMENT

Next, a fifth example embodiment of the present invention based on the above-described third example embodiment will be described.

In the following description, characteristic portions according to the present example embodiment will be mainly described and the same components as in the above-described third example embodiment are assigned with the same reference signs, whereby overlapping description will be omitted.

With reference to FIG. 23, a configuration of a blood pressure measurement device 5007 according to the fifth example embodiment of the present invention and processing in the blood pressure measurement device 5007 will be described. FIG. 23 is a block diagram illustrating the configuration of the blood pressure measurement device 5007 according to the fifth example embodiment of the present invention.

A blood pressure measurement device 5007 according to the fifth example embodiment includes a first blood pressure estimation unit 5004, a pulse wave signal generation unit 5002, a pulse wave calculation unit 5003, a blood pressure information generation unit 5005, a pressure signal generation unit 5001, and a second blood pressure estimation unit 5006. Further, the blood pressure measurement device 5007 includes a pressure measurement unit 407, a cuff 401, a pressure control unit 404, a pulse wave signal generation unit 5002, an input unit 405, and a display unit 406. A pulse wave measurement unit 402 is set on or in the cuff 401.

The blood pressure measurement device 5007 roughly processes in accordance with a “first measurement mode”, a “second measurement mode” or both the modes. The first measurement mode is a processing mode for generating blood pressure information where pulse wave information and blood pressure value are associated. The second measurement mode is a processing mode for measuring blood pressure based on blood pressure information. The input unit 405 has, for example, buttons (a button 5008 and a button 5009) that can designate the first measurement mode, the second measurement mode, or both the modes. When either button or both the buttons are pushed, the input unit 405 receives processing in accordance with the pushed button(s).

When the button 5008 to direct the first measurement mode is pushed at the input unit 405, the blood pressure measurement device 5007 executes processing in accordance with the first measurement mode. When the button 5009 to direct the second measurement mode is pushed at the input unit 405, the blood pressure measurement device 5007 executes processing in accordance with the second measurement mode. When both the buttons, the button 5008 to direct the first measurement mode and the button 5009 to direct the second measurement mode, are pushed, the blood pressure measurement device 5007 executes both processing in accordance with the first measurement mode and processing in accordance with the second measurement mode.

The pressure signal generation unit 5001 generates pressure signal that is an internal pressure of the cuff 401 measured during particular period by the pressure measurement unit 407.

In the example embodiments as described above, the pressure measurement unit 407 measures pulse wave signal. However, it is assumed that the pressure signal generation unit 5001 generates a pressure signal based on pressure measured by the pressure measurement unit 407 in each of the following example embodiments.

The pulse wave signal generation unit 5002 generates pulse wave signal representing pulse wave measured during the particular period by the pulse wave measurement unit 402

In the above example embodiments, the pulse wave measurement unit 402 measures pulse wave signal. However, it is assumed that the pulse wave signal generation unit 5002 generates pulse wave signal based on pulse wave measured by the pulse wave measurement unit 402 in each of the following example embodiments.

The first blood pressure estimation unit 5004 has, for example, functions of a blood pressure estimation unit (the blood pressure estimation unit 103 and so on) according to each example embodiment of the present invention. The first blood pressure estimation unit 5004, for example, estimates blood pressure when the second measurement mode is directed.

The second blood pressure estimation unit 5006, for example, detects sound relating to blood flow based on the Korotkoff method and estimates pressure at a timing when the sound begins due to obstruction to the blood flow as a diastolic blood pressure. The second blood pressure estimation unit 5006, for example, estimates a pressure at a timing when the sound disappears due to suspension of the blood flow as a systolic blood pressure. The second blood pressure estimation unit 5006, for example, estimates diastolic blood pressure, systolic blood pressure, or both the blood pressures based on the oscillometric method. The second blood pressure estimation unit 5006, for example, estimates blood pressure when the first measurement mode is directed.

For example, when the first measurement mode is directed, the pressure control unit 404 controls internal pressure of the cuff 401 so that the pressure is equal or more than systolic blood pressure. The systolic blood pressure is, for example, pressure measured in accordance with the Korotkoff method or the oscillometric method as described above. The pressure control unit 404 decreases internal pressure of the cuff by releasing gas (or liquid) from the cuff 401. The second blood pressure estimation unit 5006 estimates the systolic blood pressure, the diastolic blood pressure, or both the blood pressures, for example, in accordance with the Korotkoff method, or the oscillometric method.

The pulse wave calculation unit 5003 calculates a plurality of timings when the pulse wave signal satisfies predetermined conditions. The pulse wave calculation unit 5003 calculates a period between the plurality of timings (in other word, pulse wave parameters) and generates pulse wave information where the calculated pulse wave parameters and the pressures measured in the calculated period are associated.

The blood pressure information generation unit 5005 generates blood pressure information where pulse wave information, in which the pulse wave parameters and the pressure during the period represented by the pulse wave parameter are associated, and the blood pressure, for example, estimated by the second blood pressure estimation unit 5006 are associated with each other.

The first blood pressure estimation unit 5004 calculates a similarity degree that is extent of similarity between, for example, the pulse wave information generated based on the pulse wave signal and the pressure signal measured during the second period, and pulse wave information in the blood pressure information generated by the blood pressure information generation unit 5005. The first blood pressure estimation unit 5004, for example, specifies blood pressure information including pulse wave signal having the maximum (or substantially maximum) similarity degree and estimates blood pressure in the specified blood pressure information as blood pressure during the second period. Processing of the first blood pressure estimation unit 5004 will be described in detail in a sixth example embodiment of the present invention.

Referring to FIG. 24, processing in accordance with the first measurement mode in the blood pressure measurement device 5007 will be described. FIG. 25 is a flowchart illustrating a flow of processing in the blood pressure measurement device 5007 according to the fifth example embodiment when the first measurement mode is directed.

The pressure control unit 404 determines whether or not internal pressure of the cuff 401 is equal or more than systolic blood pressure (step S5001). When internal pressure of the cuff 401 is less than the systolic blood pressure (NO at step S5001), the pressure control unit 404 increase the internal pressure of the cuff 401, for example, by letting gas (or liquid) into the cuff 401 (step S5002). The pulse wave signal generation unit 5002 generates pulse wave signal where pulse wave measured while the pressure control unit 404 increases internal pressure of the cuff 401 and timing when the pulse wave is measured (step S5003).

When internal pressure of the cuff 401 is equal or more than the systolic blood pressure (YES at step S5001), the pressure control unit 404 decreases internal pressure of the cuff 401, for example, by releasing gas (or liquid) from the cuff 401 (step S5004).

The pressure measurement unit 407 measures internal pressure of the cuff 401 after blood pressure estimation begins and until the estimation is completed. The pulse wave measurement unit 402 measures pulse wave on a specific region after the blood pressure estimation begins and until the estimation is completed. The pulse wave calculation unit 5003 calculate a plurality of timings when the pulse wave signal satisfies the predetermined conditions. The pulse wave calculation unit 5003 calculates a period (in other word, a pulse wave parameter) between the calculated timings (step S5005). The pulse wave calculation unit 5003 generates pulse wave information where the calculated pulse wave parameter and the measured pressure during the calculated period are associated (step S5006).

The second blood pressure estimation unit 5006 estimates blood pressure on the specific region where the cuff 401 is set while the pressure control unit 404 increases internal pressure of the cuff 401 in accordance with, for example, the Korotkoff method or the oscillometric method. In this case, the second blood pressure estimation unit 5006 estimates systolic blood pressure, diastolic blood pressure, or both.

The blood pressure information generation unit 5005 generates blood pressure information where the blood pressure calculated by the second blood pressure estimation unit 5006 and the calculated pulse wave information are associated (step S5008). The generated blood pressure information is, for example, referred when the first blood pressure estimation unit 5004 estimates blood pressure.

Further, the pressure control unit 404 decreases internal pressure of the cuff 401 by, for example, releasing gas (or liquid) from the cuff 401.

In the flowchart in FIG. 24, processing of estimating blood pressure (step S5007) and processing of generating pulse wave information (step S5005 and step S5006) are executed sequentially. However, the processing of estimating blood pressure and the processing of generating pulse wave information may be executed in parallel (or in pseud-parallel).

Therefore, when the first measurement mode is directed, the blood pressure measurement device 5007 estimates blood pressure and generates blood pressure information where pulse wave information for the measured pressure and pulse wave, and the blood pressure for the measured pressure and pulse wave are associated with each other.

The blood pressure measurement device 5007 may generate blood pressure information where the pulse wave information, the blood pressure, and an identifier identifying each measurement subject of pressure and pulse wave are associated.

For example, when blood pressure information includes the identifier, the input unit 405 may include a user button (not depicted) associated with each identifier of the subject to be measured.

The blood pressure measurement device 5007 reads blood pressure information associated with a pushed user button, for example, under the second measurement mode. For example, the read blood pressure information is blood pressure information for the subject identified by the identifier. The blood pressure measurement device 5007 estimates blood pressure for the subject identified by the identifier based on the read blood pressure information.

Subsequently, effects on the blood pressure measurement device 5007 according to the fifth example embodiment will be explained.

The blood pressure measurement device 5007 according to the fifth example embodiment can estimate blood pressure with high accuracy. The reason for this is that the blood pressure measurement device 5007 according to the fifth example embodiment includes the blood pressure measurement device 1201 according to the third example embodiment.

The blood pressure measurement device 5007 according to the fifth example embodiment can estimate blood pressure with further high accuracy. The reason for this is that the blood pressure measurement device 5007 estimates current blood pressure for subject to be measured based on blood pressure information for himself (or herself).

Blood pressure information is generally different depending on subjects to be measured. Therefore, blood pressure information where pulse wave information for particular subject to be measured and blood pressure for the particular subject are associated is different from another blood pressure information for another subject to be measured. Therefore, blood pressure information generated in accordance with the processing described above is characteristic of each subject to be measured. The blood pressure measurement device 5007 estimates current blood pressure for the subject to be measured based on blood pressure information for himself (or herself). As a result, the blood pressure measurement device 5007 can estimate blood pressure for the subject to be measured with further high accuracy.

Moreover, the blood pressure measurement device 5007 according to the fifth example embodiment achieves high convenience of users. The reason for this will be shown in the following description. The blood pressure measurement device 5007 can execute processing corresponding to first measurement mode and processing corresponding to second measurement mode. Specifically, the blood pressure measurement device 5007 can generate blood pressure information and estimate current blood pressure based on the generated blood pressure information.

Blood pressure information may be stored in the blood pressure information generation unit 5005, the first blood pressure estimation unit 5004, or an outside storage device. The pressure measurement unit 407 may measure pressure and generate pressure signal for the measured pressure based on the measured pressure. In this case, the pressure measurement unit 407 transmits the generated pressure signal to the blood pressure information generation unit 5005. Similarly, the pulse wave measurement unit 402 may measure pulse wave and generate pulse wave signal for the measured pulse wave on the basis of the measured pulse wave. In this case, the pulse wave measurement unit 402 transmits the generated pulse wave signal to the blood pressure information generation unit 5005.

### SIXTH EXAMPLE EMBODIMENT

Next, a sixth example embodiment of the present invention based on the above-described fifth example embodiment will be described.

In the following description, characteristic portions according to the present example embodiment will be mainly described and the same components as in the above-described fifth example embodiment and other example embodiments are assigned with the same reference signs, whereby overlapping description will be omitted.

With reference to FIG. 25, a configuration of a blood pressure measurement device according to the sixth example embodiment and processing executed by the blood pressure measurement device will be described. FIG. 25 is a block diagram illustrating the configuration of the blood pressure measurement device according to the sixth example embodiment of the present invention.

A blood pressure measurement device 6007 according to the sixth example embodiment includes a first blood pressure estimation unit 6004, a pulse wave signal generation unit 5002, a blood pressure information generation unit 5005, a pulse wave calculation unit 5003, a pressure signal generation unit 5001, and a second blood pressure estimation unit 5006. Further, the blood pressure measurement device 6007 includes a pressure measurement unit 407, a cuff 401, a pressure control unit 404, a pulse wave signal generation unit 5002, an input unit 405, and a display unit 406. A pulse wave measurement unit 402 is disposed in the cuff 401.

For convenience of explanation, it is assumed that specific pulse wave information is pulse wave information for a target for which a blood pressure is estimated. The specific pulse wave information represents pulse wave information generated by the pulse wave calculation unit 5003 on the basis of a pulse wave signal generated for a pulse wave measured by the pulse wave measurement unit 402.

The first blood pressure estimation unit 6004 estimates blood pressure for specific pulse wave information on the basis of blood pressure information including blood pressure estimated by the second blood pressure estimation unit 5006.

The first blood pressure estimation unit 5004 described in the fifth example embodiment estimates blood pressure on the basis of pulse wave information having the maximum (or substantially maximum) similarity degree to the specific pulse wave information in blood pressure information. In contrast, in the present example embodiment, when the first blood pressure estimation unit 6004 determines that a similarity degree does not satisfy a predetermined condition, the second blood pressure estimation unit 5006 estimates blood pressure in accordance with a Korotkoff method, an oscillometric method, or the like. In other words, the first blood pressure estimation unit 6004 estimates blood pressure when a maximum (or substantially maximum) similarity degree satisfies the predetermined condition. Otherwise, the first blood pressure estimation unit 6004 does not estimate blood pressure.

Referring to FIG. 26, a flow of processing executed by the blood pressure measurement device 6007 will be described in detail. FIG. 26 is a flowchart illustrating a flow of processing in the blood pressure measurement device 6007 according to the sixth example embodiment.

The first blood pressure estimation unit 6004 calculates a similarity degree between each piece of pulse wave information included in blood pressure information and the specific pulse wave information (step S6001). Next, the first blood pressure estimation unit 6004 specifies a maximum (or substantially maximum) similarity degree for the calculated similarity degree (step S6002). Next, the first blood pressure estimation unit 6004 determines whether or not the specified maximum (or substantially maximum) similarity degree satisfies a predetermined condition (step S6003). The predetermined condition is, for example, a condition whether or not a maximum (or substantially maximum) similarity degree exceeds a predetermined threshold. When the specified similarity degree exceeds the predetermined threshold, the calculated similarity degree satisfies the predetermined condition. Further, when the calculated similarity degree is equal to or less than the predetermined threshold, the specified similarity degree does not satisfy the predetermined condition. The predetermined condition may be a condition similar to the above-described condition and is not necessarily limited to the above-described condition.

When a similarity degree satisfies the predetermined condition (YES in step S6003), the first blood pressure estimation unit 6004 specifies blood pressure information including pulse wave information having the specified maximum (or substantially maximum) similarity degree (step S6004). Next, the pressure control unit 404 reduces internal pressure of the cuff 401 (step S6005). With regard to the step S6004 and the step S6005, the processing of the step S6004 may be executed after the processing of the step S6005 is executed.

As described in the first example embodiment, it is not always necessary for the blood pressure estimation unit 103 to calculate a similarity degree between all pieces of data of pulse wave information in blood pressure information and specific pulse wave information, and partial data of the pulse wave information in the blood pressure information are employable. Further, the pressure control unit 404 may stop increasing the pressure at a timing when a similarity degree satisfies a predetermined condition.

Next, the first blood pressure estimation unit 6004 estimates blood pressure for the specific pulse wave information on the basis of blood pressure included in the specified blood pressure information (step S6006). When number of pieces of the specified blood pressure information is one, the first blood pressure estimation unit 6004 estimates blood pressure included in the specified blood pressure information as blood pressure for the specific pulse wave information. When number of pieces of the specified blood pressure information is multiple, the first blood pressure estimation unit 6004 calculates, for example, an average value (or a median) of the respective blood pressure included in the multiple pieces of the specified blood pressure information, and estimates the calculated value as blood pressure for the specific pulse wave information.

On the other hand, when the specified similarity degree does not satisfy the predetermined condition (NO in step S6003), the processing indicated in the step S5001 to the step S5008 in FIG. 24 is executed (step S6007). In other words, the second blood pressure estimation unit 5006 estimates blood pressure in accordance with a Korotkoff method, an oscillometric method, or the like without blood pressure information. Further, by the processing indicated in the step S5001 to the step S5008, blood pressure information including pulse wave information similar to (or consistent with) the specific pulse wave information that is a target for a similarity degree calculation in the step S6001 is generated. Therefore, with regard to the pulse wave information similar to the specific pulse wave information, the blood pressure measurement device 6007 can accurately estimate blood pressure on the basis of the generated blood pressure information.

Next, advantageous effects of the blood pressure measurement device 6007 according to the sixth example embodiment will be described.

The blood pressure measurement device 6007 according to the sixth example embodiment can estimate blood pressure with a high degree of accuracy. The reason is that the blood pressure measurement device 6007 according to the sixth example embodiment includes the blood pressure measurement device 5007 according to the fifth example embodiment.

The blood pressure measurement device 6007 according to the sixth example embodiment can estimate blood pressure with a further higher degree of accuracy. One reason for this advantageous effect is that, when a similarity degree satisfies a predetermined condition, the blood pressure measurement device 6007 estimates blood pressure on the basis of blood pressure information, and, when a similarity degree does not satisfy the predetermined condition, the blood pressure measurement device 6007 estimates blood pressure in accordance with a Korotkoff method, an oscillometric method, or the like. Further, one reason for this advantageous effect is that blood pressure information including blood pressure measured when a similarity degree does not satisfy the predetermined condition is generated, and thereby, even when a similarity degree does not satisfy the predetermined condition, blood pressure information including specific pulse wave information that is a target for which the similarity degree is calculated is generated. As a result of generation of new blood pressure information, thereafter, when measured pulse wave information is similar to pulse wave information included in the new blood pressure information, the blood pressure measurement device 6007 can estimate blood pressure with a high degree of accuracy on the basis of the new blood pressure information.

These reasons will be described in detail. When a similarity degree does not satisfy a predetermined condition in the step S6003, blood pressure information does not include pulse wave information suitable for estimating blood pressure for the specific pulse wave information. In other words, in this case, even when the blood pressure measurement device 6007 specifies blood pressure information including pulse wave information similar to specific pulse wave information, pulse wave information included in the specified blood pressure information is not similar to the specific pulse wave information. Therefore, it is difficult for the first blood pressure estimation unit 6004 to accurately estimate blood pressure for the specific pulse wave information.

On the other hand, when a similarity degree does not satisfy a predetermined condition, the blood pressure measurement device 6007 generates blood pressure information in accordance with the flowchart exemplarily illustrated in FIG. 24. As a result, even when blood pressure information does not include pulse wave information similar to (or consistent with) the specific pulse wave information, the blood pressure measurement device 6007 generates blood pressure information for the specific pulse wave information. Therefore, thereafter, when measured pulse wave information is similar to (or consistent with) the pulse wave information included in the generated blood pressure information, the blood pressure measurement device 6007 according to the present example embodiment can estimate blood pressure with a high degree of accuracy on the basis of the generated blood pressure information.

Further, the blood pressure measurement device 6007 according to the sixth example embodiment may stop increasing pressure at a cuff internal pressure less than a systolic blood pressure when a similarity degree between partial pulse wave information included in blood pressure information and measured pulse wave information is high. Even in such a case, the blood pressure measurement device 6007 can estimate blood pressure with a high degree of accuracy on the basis of blood pressure information.

Therefore, the blood pressure measurement device 6007 according to the sixth example embodiment can execute both estimation processing of blood pressure and improvement processing of estimation accuracy of blood pressure. The blood pressure measurement device 6007 according to the sixth example embodiment can estimate blood pressure with a higher degree of accuracy when blood pressure is repeatedly measured for a plurality of times. In addition, the blood pressure measurement device 6007 can execute processing of generating blood pressure information and does not need to receive blood pressure information from outside. Therefore, the blood pressure measurement device 6007 according to the sixth example embodiment can realize high convenience.

(Hardware Configuration Example)

A configuration example of hardware resources that realize a blood pressure measurement device in the above-described example embodiments of the present invention using a single calculation processing apparatus (an information processing apparatus or a computer) will be described. However, the blood pressure estimation device may be realized using physically or functionally at least two calculation processing apparatuses. Further, the blood pressure estimation device may be realized as a dedicated apparatus.

FIG. 27 is a block diagram schematically illustrating a hardware configuration of a calculation processing apparatus capable of realizing each of the a blood pressure estimation devices and each of the blood pressure measurement devices according to each of the first example embodiment to the sixth example embodiment. A calculation processing apparatus 20 includes a central processing unit (CPU) 21, a memory 22, a disc 23, a non-transitory recording medium 24, an input apparatus 25, an output apparatus 26, and a communication interface (hereinafter, expressed as a “communication I/F”) 27. The calculation processing apparatus 20 can execute transmission/reception of information to/from another calculation processing apparatus and a communication apparatus via the communication I/F 27.

The non-transitory recording medium 24 is, for example, a computer-readable Compact Disc, Digital Versatile Disc. The non-transitory recording medium 24 is, for example, Universal Serial Bus (USB) memory, or Solid State Drive. The non-transitory recording medium 24 allows a related program to be holdable and portable without power supply. The non-transitory recording medium 24 is not limited to the above-described media. Further, a related program can be carried via a communication network by way of the communication I/F 27 instead of the non-transitory recording medium 24.

In other words, the CPU 21 copies, on the memory 22, a software program (a computer program: hereinafter, referred to simply as a “program”) stored by the disc 23 when executing the program and executes arithmetic processing. The CPU 21 reads data necessary for program execution from the memory 22. When display is needed, the CPU 21 displays an output result on the output apparatus 26. When a program is input from the outside, the CPU 21 reads the program from the input apparatus 25. The CPU 21 interprets and executes a blood pressure estimation program present on the memory 22 corresponding to a function (processing) indicated by each unit illustrated in FIG. 1, FIG. 7, FIG. 10, FIG. 20, FIG. 22, FIG. 24, or FIG. 26 described above or a blood pressure estimation program (FIG. 2, FIG. 11, FIG. 21, FIG. 25, or FIG. 27). The CPU 21 sequentially executes the processing described in each example embodiment of the present invention.

In other words, in such a case, it is conceivable that the present invention can also be made using the blood pressure estimation program. Further, it is conceivable that the present invention can also be made using a computer-readable, non-transitory recording medium storing the blood pressure estimation program.

The present invention has been described using the above-described example embodiments as exemplary cases. However, the present invention is not limited to the above-described example embodiments. In other words, the present invention is applicable with various aspects that can be understood by those skilled in the art without departing from the scope of the present invention.

This application is based upon and claims the benefit of priority from Japanese patent application No. 2015-108033, filed on May 28, 2015, the disclosure of which is incorporated herein in its entirety.

### REFERENCE SIGNS LIST

101 Blood pressure estimation device
102 Pulse wave calculation unit
103 Blood pressure estimation unit
2001 Pulse wave signal
2003 Pressure signal
401 Cuff
402 Pulse wave measurement unit
404 Pressure control unit
405 Input unit
406 Display unit
407 Pressure measurement unit
408 Blood pressure measurement device
901 Blood pressure estimation device
902 Pulse wave calculation unit
903 Blood pressure estimation unit
1101 Skin
1102 Subcutaneous tissue
1103 Artery wall
1104 Blood flow
1105 Obstacle
a State
b State
81 First timing
82 Second timing
83 Third timing
84 Fourth timing
85 Fifth timing
86 Sixth timing
1581 First curve
1582 Second curve
1583 Third curve
1585 Fifth curve
1586 Sixth curve
1001 Pulse wave measurement unit
1002 Pulse wave measurement unit
1003 Pulse wave measurement unit
1004 Pulse wave measurement unit
1005 Cuff
1006 Pressure bag
1007 Blood pressure measurement device
1008 Blood pressure measurement device
1201 Blood pressure measurement device
1202 Blood pressure estimation device
1203 Pressure control unit
2501 Blood pressure estimation device
2502 Determination unit
2503 Correction unit
1402 Blood pressure estimation device
20 Computing device
21 CPU
22 Memory
23 Disk
24 Non-transitory recording medium
25 Input device
26 Output device
27 Communication IF
5001 Pressure signal generation unit
5002 Pulse wave signal generation unit
5003 Pulse wave calculation unit
5004 First blood pressure estimation unit
5005 Blood pressure information generation unit
5006 Second blood pressure estimation unit
5007 Second blood pressure estimation unit
5008 Button
5009 Button
6004 First blood pressure estimation unit
6007 Blood pressure measurement device

What is claimed is:

1. A blood pressure measurement device comprising:
a first blood pressure estimation unit configured to estimate blood pressure on basis of first pressure signal indicating internal pressure of cuff during a first period and Korotkoff sound during the first period or on basis of the first pressure signal during the first period and first pulse wave signal indicating pulse wave during the first period;
a pulse wave calculation unit configured to calculate a plurality of timings when the first pulse wave signal satisfies a predetermined condition, a third period between the plurality of timings, and pressure of first pressure signal during the third period and generate first pulse wave information where the third period and the pressure are associated;
a blood pressure information generation unit configured to generate blood pressure information where the calculated first pulse wave information and the estimated blood pressure are associated with each other; and
a second blood pressure estimation unit configured to specify the first pulse wave information matching or resembling second pulse wave information generated based on second pressure signal indicating internal pressure of the cuff during second period and second pulse wave signal indicating pulse wave during the second period, and estimate the blood pressure associated with the specified first pulse wave information as blood pressure during the second period.

2. The blood pressure measurement device according to claim 1, further comprising:
a pressure control unit configured to control the internal pressure so as to make the maximum of the internal pressure during the first period equal to or larger than systolic blood pressure during the first period, and control the internal pressure so as to make the maximum of the internal pressure during the second period smaller than systolic blood pressure during the second period.

3. The blood pressure measurement device according to claim 1, wherein,
the first blood pressure estimation unit estimates the blood pressure when the second blood pressure estimation unit determines that the second pulse wave information does not match and does not resemble the first pulse wave information in the blood pressure information.

4. The blood pressure measurement device according to claim 3, wherein,
when the second blood pressure estimation unit determines that the second pulse wave information during the second period does not match and does not resemble the first pulse wave information in the blood pressure information, the pulse wave calculation unit generates the first pulse wave information and the blood pressure information generation unit generates the blood pressure information including the generated first pulse wave information.

5. The blood pressure measurement device according to claim 1, wherein,
the predetermined condition is whether or not the first pulse wave signal is minimal or substantially minimal in a pulse wave, and
the pulse wave calculation unit generates the first pulse wave information on a case of satisfying the predetermined condition.

6. The blood pressure measurement device according to claim 1, wherein,
the predetermined condition is a first condition of whether the first pulse wave signal or a derived signal representing N-order discrete differential or N-dimensional differential (N is natural number) of the first pulse wave signal takes particular value, and
the pulse wave calculation unit calculates, on basis of the predetermined condition, the first pulse wave information on a case when the first pulse wave signal or the derived signal takes the particular value.

7. The blood pressure measurement device according to claim 6, wherein,
the predetermined condition is a combination of a plurality of the first condition, and
the pulse wave calculation unit calculates the first pulse wave information on a case of satisfying the predetermined condition.

8. The blood pressure measurement device according to claim 1, wherein,
the pulse wave calculation unit calculates the third period between a timing when a particular character is occurred in a pulse wave and a timing in the plurality of timings.

9. A blood pressure measurement method by a blood pressure measurement device including a cuff which can store gas or liquid, a pulse wave measurement unit configured to measure pulse wave, and a pressure measurement unit configured to measure internal pressure of the cuff comprising:
estimating blood pressure on basis of first pressure signal indicating internal pressure of cuff during a first period and Korotkoff sound during the first period or on basis of the first pressure signal during the first period and first pulse wave signal indicating pulse wave during the first period;
calculating a plurality of timings when the first pulse wave signal satisfies a predetermined condition, a third period between the plurality of timings, and pressure of first pressure signal during the third period and generating first pulse wave information where the third period and the pressure are associated;
generating blood pressure information where the calculated first pulse wave information and the estimated blood pressure are associated with each other; and
specifying the first pulse wave information matching or resembling second pulse wave information generated based on second pressure signal indicating internal pressure of the cuff during second period and second pulse wave signal indicating pulse wave during the second period, and estimating the blood pressure associated with the specified first pulse wave information as blood pressure during the second period.

10. A non-volatile recording medium recording a blood pressure measurement program, for a blood pressure measurement device including a cuff which can store gas or liquid, a pulse wave measurement unit configured to measure pulse wave, and a pressure measurement unit configured to measure internal pressure of the cuff, that causes a computer to realize:
a first blood pressure function configured to estimate blood pressure on basis of first pressure signal indicating internal pressure of cuff during a first period and Korotkoff sound during the first period or on basis of the first pressure signal during the first period and first pulse wave signal indicating pulse wave during the first period;
a pulse wave calculation function configured to calculate a plurality of timings when the first pulse wave signal satisfies a predetermined condition, a third period between the plurality of timings, and pressure of first pressure signal during the third period and generate first pulse wave information where the third period and the pressure are associated;
a blood pressure information generation function configured to generate blood pressure information where the calculated first pulse wave information and the estimated blood pressure are associated with each other; and
a second blood pressure estimation function configured to specify the first pulse wave information matching or resembling second pulse wave information generated based on second pressure signal indicating internal pressure of the cuff during second period and second pulse wave signal indicating pulse wave during the second period, and estimate the blood pressure associated with the specified first pulse wave information as blood pressure during the second period.
